(19) United States
(12) Patent Application Publication
(10) Pub. No.: US 2026/0286289 A1
(43) Pub. Date: Sep. 24, 2026

(54) CELL STORAGE AND TRANSPORTATION MEDIUM, SYSTEM, AND METHOD OF CELL AGGREGATES

(71) Applicant: CORNING INCORPORATED, CORNING, NY (US)

(72) Inventors: Gregory Roger Martin, Acton, ME (US); Ana Maria del Pilar Pardo, Portsmouth, NH (US); Allison Jean Tanner, Bowdoinham, ME (US)

(21) Appl. No.: 19/685,393

(22) Filed: May 22, 2026

Related U.S. Application Data
(62) Division of application No. 17/613,994, filed on May 22, 2020.
(60) Provisional application No. 62/854,556, filed on May 30, 2019.

(51) Int. Cl. C12N 5/00; C12M 1/32

(52) U.S. Cl. CPC C12N 5/0062; C12M 23/12; C12N 2533/76; C12N 2533/78; C12N 2539/10

(57) ABSTRACT

The present disclosure relates to agarose and methylcellulose storage and transport mediums, systems for cell storage and transport, and cell storage and transport methods. The instantly-disclosed agarose and methylcellulose storage and transport medium is ideally suited for 3D spheroid cell culture storage and transport. In particular aspects, the storage and transport mediums, systems, and methods are used in combination with or performed in labware that combine 3D spheroid culture with a gas permeable, micro-patterned design that allows for protection and prolonged maintenance of spheroid cell (e.g. hepatocytes) viability and functionality during storage and transport.

### CROSS REFERENCE TO RELATED APPLICATIONS

This Application is a divisional of U.S. Patent Application Serial No. 17/613,994 filed on Nov. 24, 2021, which is a national stage application under 35 U.S.C. §371 of International Application No. PCT/US2020/034120, filed May 22, 2020, which claims the benefit of priority under 35 U.S.C §120 of U.S. Provisional Application Serial No. 62/854,556 filed on May 30, 2019, the content of which is relied upon and incorporated herein by reference in its entirety.

### FIELD

The present disclosure generally relates to agarose and methylcellulose cell storage and transport mediums, systems for cell storage and transport, and cell storage and transport methods. In particular aspects, the transport mediums, systems, and methods are used in combination with or performed in labware that combine 3D spheroid or organoid culture with a gas permeable, micro-patterned design that allows for protection and prolonged maintenance of spheroid cell (e.g. hepatocytes) viability and functionality during storage and transport.

### TECHNICAL BACKGROUND

Cells cultured in three dimensions, such as spheroids, can exhibit more in vivo like functionality than their counterparts cultured in two dimensions as monolayers. Particularly, cells cultured in three dimensions more closely resemble in vivo tissue in terms of cellular communication and the development of extracellular matrices. Thus, there is an increasing demand for the storage and transportation of 3D cultured cells, such as spheroids, for various cell culture tests and examinations in numerous biotechnology-related fields. However, storage and transportation of 3D cultured cells, including spheroids, remains a challenge. In two-dimensional cell culture systems, cells can attach to a substrate on which they are cultured. However, when cells are grown in three dimensions, such as spheroids or organoids, the cells interact with each other rather than attaching to the substrate, making such cells more susceptible to cell viability damage and integrity damage, and even cell death, due to disturbances during the storage and transportation process. Thus, it is difficult to transport stored cells cultured in three dimensions, such as spheroids. Agarose has been used as a support for 3D cell culture because it is a non-binding surface. The retrieval of cells in agarose typically involves melting the agarose at temperatures cells cannot withstand, causing cell viability damage and even death to the cultured cells. Further, use of agaroses in transport mediums for culture systems often requires the use of agarase and additional steps for facilitating the removal of the agarose and thus allowing the release of the cells forming the transported culture.

Accordingly, on-going need exists for alternative transport mediums, systems for cell storage and transport, and cell storage and transport methods, and more particularly for alternative transport mediums, systems for cell storage and transport, and cell storage and transport methods that allow for protection and prolonged maintenance of spheroid or organoid cell viability and functionality during storage and transport.

### SUMMARY OF THE DISCLOSURE

In accordance with various embodiments of the present disclosure, agarose and methylcellulose transport mediums, systems for cell storage and transport, and cell storage and transport methods are disclosed. In particular aspects, the transport mediums, systems and methods are used in combination with or performed in labware that combine 3D spheroid or organoid culture with a gas permeable, micro-patterned design that allows for protection and prolonged maintenance of spheroid cell (e.g. hepatocytes) viability and functionality during storage and transport.

In various embodiments, a cell storage and transport medium is disclosed. In aspects, the cell storage and transport medium comprises a mixture of cell culture medium, agarose and methylcellulose, wherein the final agarose concentration in the storage and transport medium is about 0.5 to about 1.0% and the final methylcellulose concentration in the storage and transport medium is about 0.5 to about 0.7%.

In some aspects of a cell storage and transport medium, the agarose is an ultra-low gelling temperature agarose (which may be abbreviated as “ARG-L”). In some aspects, the agarose has a gelling temperature of 8 - 17° C. In some aspects, the cell storage and transport medium is a firm gel at 4° C. In some aspect, the cell storage and transport medium is a soft gel at 23° C. In some aspects, the cell storage and transport medium is a viscous liquid at 37° C.

In various embodiments, a cell storage and transportation system is disclosed. In aspects, the system comprises: cells; a cell culture article, wherein the cell culture article comprises a chamber, the chamber comprising an array of microcavities, each microcavity structured to constrain the cells to grow in a 3D spheroid or organoid confirmation; and a cell storage and transport medium comprising a mixture of agarose and methylcellulose, wherein the final agarose concentration in the storage and transport medium is about 0.5 to about 1.0% and the final methylcellulose concentration in the storage and transport medium is about 0.5 to about 0.7%.

In aspects of a cell storage and transportation system, each microcavity of the chamber of the cell culture article includes a top aperture and a liquid impermeable bottom comprising a bottom surface. In embodiments, at least a portion of the bottom surface includes a low-adhesion or no-adhesion material in or on the bottom surface. In some embodiments, the liquid impermeable bottom including the bottom surface is gas-permeable. In some embodiments, at least a portion of the bottom is transparent.

In aspects of a cell storage and transportation system, the bottom surface comprises a concave bottom surface. In some embodiments, the at least one concave surface of each microcavity of the chamber includes a hemi-spherical surface, a conical surface having a taper of 30 to about 60 degrees from the side walls to the bottom surface, or a combination thereof.

In aspects of a cell storage and transportation system, the chamber further comprises a side wall. In some embodiments, the side wall surface of each microcavity of the chamber includes a vertical cylinder, a portion of a vertical conic of decreasing diameter from the chamber’s top to bottom surface, a vertical square shaft having a conical transition to the at least one concave bottom surface, or a combination thereof.

In aspects of a cell storage and transportation system, the cell culture article further includes a chamber annex for receiving a pipette tip for aspiration, the chamber annex including a surface adjacent to and in fluid communication with the chamber, the chamber annex having a second bottom spaced away and at an elevation above the bottom surface, wherein the second bottom deflects fluid dispensed from a pipette away from the bottom surface.

In aspects of a cell storage and transportation system, the cell culture article includes from 1 to about 2,000 of said chambers, wherein each chamber is physically separated from any other chamber. In some embodiments, each chamber includes from about 1 to about 800 per square centimeter of said microcavities.

In aspects of a cell storage and transportation system, the agarose of the cell storage and transport medium is an ultra-low gelling temperature agarose. In some aspects of a cell storage and transportation system, the agarose has a gelling temperature of 8 - 17° C. In some aspects, the cell storage and transport medium is a firm gel at 4° C. In some aspects of a cell storage and transportation system, the cell storage and transport medium is a soft gel at 23 ° C. In some aspects of a cell storage and transportation system, the cell storage and transport medium is a viscous liquid at 37° C.

In various embodiments, a method for the transport of cells is disclosed. In aspects, a method for the transport of cells includes a) culturing live cells in a cell culture article to form a spheroid, wherein the cell culture article comprises a chamber, the chamber comprising an array of microcavities, each microcavity structured to constrain the cells to grow in a 3D spheroid or organoid confirmation; b) adding a cell storage and transport medium comprising a mixture of cell culture medium, agarose and methylcellulose, wherein the final agarose concentration in the storage and transport medium is 0.5 to 1.0% and the final methylcellulose concentration in the storage and transport medium is 0.5 to 0.7% to the cell culture; c) solidifying the cell storage and transport medium; and d) transporting the cell culture article.

In aspects of a method for the transport of cells, each microcavity of the chamber of the cell culture article includes a top aperture and a liquid impermeable bottom comprising a bottom surface. In aspects, at least a portion of the bottom surface includes a low-adhesion or no-adhesion material in or on the bottom surface. In some aspects, the liquid impermeable bottom including the bottom surface is gas-permeable. In some aspects, at least a portion of the bottom is transparent.

In aspects of a method for the transport of cells, the bottom surface comprises a concave bottom surface. In some aspects, the at least one concave surface of each microcavity of the chamber includes a hemi-spherical surface, a conical surface having a taper of 30 to about 60 degrees from the side walls to the bottom surface, or a combination thereof.

In aspects of a method for the transport of cells, the chamber further comprises a side wall. In some embodiments, the side wall surface of each microcavity of the chamber includes a vertical cylinder, a portion of a vertical conic of decreasing diameter from the chamber’s top to bottom surface, a vertical square shaft having a conical transition to the at least one concave bottom surface, or a combination thereof.

In aspects of a method for the transport of cells, the cell culture article further includes a chamber annex for receiving a pipette tip for aspiration, the chamber annex including a surface adjacent to and in fluid communication with the chamber, the chamber annex having a second bottom spaced away and at an elevation above the bottom surface, wherein the second bottom deflects fluid dispensed rom a pipette away from the bottom surface.

In aspects of a method form the transport of cells, the cell culture article includes from 1 to about 2,000 of said chambers, wherein each chamber is physically separated from any other chamber. In some embodiments, each chamber includes from about 1 to about 800 of said microcavities per square centimeter.

In aspects of a method for the transport of cells, the agarose of the cell storage and transport medium is an ultra-low gelling temperature agarose. In some aspects of a cell storage and transportation system, the agarose has a gelling temperature of 8 - 17° C. In some aspects, the cell storage and transport medium is a firm gel at 4° C. In some aspects of a cell storage and transportation system, the cell storage and transport medium is a soft gel at 23° C. In some aspects of a cell storage and transportation system, the cell storage and transport medium is a viscous liquid at 37° C.

In aspects of a method for the transport of cells, b) comprises adding cell storage and transport medium to the cells in culture at 37° C.

In aspects of a method for the transport of cells, c) is carried out at a temperature of about 4° C or less. In aspects of a method for the transport of cells, d) is carried out at a temperature of about 4° C or less.

In aspects of a method for the transport of cells, the transport time is not more than 48 or 72 hours.

In aspects of a method of the transport of cells, the method further includes sealing the cell culture chamber before transport.

In aspects of a method of the transport of cells, the method further includes e) recovery of the transported cells. In aspects, e) comprises removing the cell storage and transport medium and replacing it with culture medium. In aspects, e) includes incubating the cell culture article at about 37° C for at least about 1 hr; and subsequently removing the cell storage and transport medium and replacing it with culture medium. In aspects, e) includes adding cell culture media that is about 37° C to the cell storage and transport medium; incubating the cell culture article at about 37° C for at least about 1 hr; and removing the cell storage and transport medium and replacing it with culture medium. In aspects, e) includes incubating the cell culture article at about 37° C for at least about 1 hr; and subsequently removing the cell storage and transport medium and extracting the 3D spheroid or organoid cells from the cell culture article.

Additional features and advantages of the subject matter of the present disclosure will be set forth in the detailed description which follows, and in part will be readily apparent to those skilled in the art from that description or recognized by practicing the subject matter of the present disclosure as described herein, including the detailed description which follows, the claims, as well as the appended drawings.

It is to be understood that both the foregoing general description and the following detailed description present embodiments of the subject matter of the present disclosure, and are intended to provide an overview or framework for understanding the nature and character of the subject matter of the present disclosure as it is claimed. The accompanying drawings are included to provide a further understanding of the subject matter of the present disclosure, and are incorporated into and constitute a part of this specification. The drawings illustrate various embodiments of the subject matter of the present disclosure and together with the description serve to explain the principles and operations of the subject matter of the present disclosure. Additionally, the drawings and descriptions are meant to be merely illustrative, and are not intended to limit the scope of the claims in any manner.

### DESCRIPTION OF THE FIGURES

The following detailed description of specific embodiments of the present disclosure can be best understood when read in conjunction with the following drawings, where like structure is indicated with like reference numerals and in which:

FIGS. 1A, 1B and 1C show an embodiment of a multi-well microplate, in this case a 96-well spheroid microplate, having an array of microcavities on the bottom surface of each well to provide multiple spheroids in each of the 96 wells. FIG. 1A shows a multi-well microplate. FIG. 1B shows illustrates a single well of the multi-well plate. FIG. 1C is an exploded view of the area of the bottom surface of the single well shown in the box C in FIG. 1B.

FIG. 2A is an illustration of an exemplary array of microcavities. FIG. 2B is an illustration of an additional exemplary array of microcavities.

FIG. 3 is an illustration showing a microcavity insert.

FIG. 4 is a photograph showing the consistency of embodiments of the cell storage and transport medium at different temperatures, from the top of the image: At 4 °C, the consistency of the transport medium is a firm gel; at room temperature (RT, 23 °C), consistency of the transport medium is a soft gel; and at 37 °C, the transport medium is a viscous liquid. This is detectable in the images by the depth of the transport medium (pink material) in the tubes held at a slight angle which permits the gel to move toward the lower end of the tube. The cell storage and transport medium is a combination of cell culture medium with ultra-low gelling temperature agarose (AGR-L) and methylcellulose (Mc).

FIGS. 5A-D are images of cell storage and transport medium formulations at room temperature to evaluate consistency of solutions as storage temperature changed from 4° C to room temperature. FIG. 5A shows a 1% AGR-L/1% Mc formulation. FIG. 5B shows a 1% AGR-L/0.7% Mc formulation. FIG. 5C shows a 1% AGR-L/0.5% Mc formulation. FIG. 5D shows a 1% AGR-L/0.35% Mc formulation.

FIG. 6 shows images of1% AGR-L cell storage and transport medium formulations in 60mm dishes after being allowed to reach room temperature. FIG. 6 shows, from left to right, 1% AGR-L/0.35% Mc,1% AGR-L/0.5% Mc, 1% AGR-L/0.7% Mc, and 1% AGR-L/1% Mc. Dishes were tipped forward to demonstrate consistency of cell storage and transport medium formulations at room temperature.

FIGS. 7A-D are images of cell storage and transport medium formulations after cold storage at 4° C. FIG. 7A shows a 0.5% AGR-L/1% Mc formulation. FIG. 7B shows a 0.5% AGR-L/0.7% Mc formulation. FIG. 7C shows a 0.5% AGR-L/0.5% Mc formulation. FIG. 7D shows a 0.5% AGR-L/0.35% Mc formulation.

FIG. 8 shows images of 0.5% AGR-L cell storage and transport medium formulations in 60mm dishes after being allowed to reach room temperature. FIG. 8 shows, from left to right, 0.5% AGR-L/0.35% Mc, 0.5% AGR-L/0.5% Mc, 0.5% AGR-L/0.7% Mc, and 0.5% AGR-L/1.0% Mc. Dishes were tipped forward to demonstrate consistency of cell storage and transport medium formulations at room temperature.

FIG. 9 show images of 60mm dishes with residual cell storage and transport medium formulation material after three dilution/ heat cycles and removal of liquefied/softened material. FIG. 9 shows dishes covered for identification purposes, the samples on the top rows, from left to right, include 1.0% AGR-L/1% Mc, 1.0% AGR-L/0.7% Mc,1.0% AGR-L/0.5 % Mc, and1.0% AGR-L/0.35% Mc, while the samples on the bottom rows, from left to right, include 0.5% AGR-L/1.0% Mc, 0.5% AGR-L/0.7% Mc, 0.5% AGR-L/0.5% Mc, and0.5% AGR-L/0.35% Mc.

FIGS. 10A-C show images of HT-29 spheroid cultures (2x magnification) for 1% AGR-L/ 0.7% Mc formulation (control). FIG. 10A shows images of cultures after 4° C storage remained in place. FIG. 10B shows images after three dilution cycles, about 20% of TM remained in vessel in the form of a gel like material covering the surface. After cell storage and transport medium removal, spheroids appear somewhat irregular. FIG. 10C shows an image of cultures 24 hr after removal of cell storage and transport medium that are in a recovery period.

FIGS. 11A-C show images of HT-29 spheroid cultures (2x magnification) for 0.5% AGR-L/ 0.7% Mc formulation. FIG. 11A shows images of cultures after 4° C storage remained in place. FIG. 11B shows images after 99% of the cell storage and transport medium was removed from the culture vessel after three dilution cycles, with minimal spheroid loss. FIG. 11C shows an image of cultures 24 hr after removal of cell storage and transport medium that are in a recovery period.

FIGS. 12A-C show images of HT-29 spheroid cultures (2x magnification) for 0.5% AGR-L/ 0.5% Mc formulation. FIG. 12A shows images of cultures after 4° C storage and that some spheroids had been dislodged from the microcavities. FIG. 12B shows images after 99% of the cell storage and transport medium was removed from the culture vessel after three dilution cycles, with minimal spheroid loss. FIG. 12C shows an image of cultures 24 hr after removal of cell storage and transport medium that are in a recovery period. Trace amounts of the cell storage and transport media was observed in microcavities.

FIGS. 13A-C show images of HT-29 spheroid cultures (2x magnification) for 0.5% AGR-L/0.35% Mc formulation. FIG. 13A shows images of cultures after 4° C. FIG. 13B shows images after 99% of the cell storage and transport medium was removed from the culture vessel after three dilution cycles, with significant spheroid loss. FIG. 13C shows an image of cultures 24 hr after removal of cell storage and transport medium that are in a recovery period.

FIG. 14 shows the results related to spheroid health. Spheroid cell health was monitored through spheroid growth (size measurements). Size measurements were taken before cell storage and transport medium formulations addition, after storage at 4° C and 48 hr. post ship test evaluation and removal of cell storage and transport medium formulations. Measurements (FIG. 14) indicate no negative side effects (spheroid dissociation or change in size) after cold storage or during recovery stage.

FIG. 15A-D are images of HT-29 cells labeled with green fluorescent protein (GFP). FIG. 15A shows the cells in normal cell culture growth medium of McCoy’s with 10% FBS. FIG. 15B shows the cells in cell storage and transport medium (in this instance, 0.5% ultra-low gel temperature agarose /0.7% R&D Systems methylcellulose medium) after storage at 4° C for 24 hours. FIG. 15C shows the cells 24 hours after the cell storage and transport medium has been removed and replaced with growth medium. FIG. 15D shows the cells 48 hours after the cell storage and transport medium has been removed and the cells have been stained with propidium iodide to detect cell death. No dead cells were detected.

FIG. 16 is flow chart for the mock transportation test process which details conditions used to assess the cell storage and transport medium (in this instance, 0.5% ultra-low gel temperature agarose /0.7% R&D Systems methylcellulose medium).

### DETAILED DESCRIPTION

Reference will now be made in greater detail to various embodiments of the subject matter of the present disclosure, some embodiments of which are illustrated in the accompanying drawings. Like numbers used in the figures refer to like components, steps and the like. However, it will be understood that the use of a number to refer to a component in a given figure is not intended to limit the component in another figure labeled with the same number. In addition, the use of different numbers to refer to components is not intended to indicate that the different numbered components cannot be the same or similar to other numbered components.

The following description of particular embodiment(s) is merely exemplary in nature and is in no way intended to limit the scope of the invention, its application, or uses, which may, of course, vary. The invention is described with relation to the non-limiting definitions and terminology included herein. These definitions and terminology are not designed to function as a limitation on the scope or practice of the invention but are presented for illustrative and descriptive purposes only. Unless otherwise defined, all terms (including technical and scientific terms) used herein have the same meaning as commonly understood by one of ordinary skill in the art to which this disclosure belongs. It will be further understood that terms such as those defined in commonly used dictionaries, should be interpreted as having a meaning that is consistent with their meaning in the context of the relevant art and the present disclosure, and will not be interpreted in an idealized or overly formal sense unless expressly so defined herein.

### DEFINITIONS

As used herein, singular forms “a,” “an” and “the” include plural referents unless the context clearly dictates otherwise. Thus, for example, reference to a “structured bottom surface” includes examples having two or more such “structured bottom surfaces” unless the context clearly indicates otherwise.

As used in this specification and the appended claims, the term “or” is generally employed in its sense including “and/or” unless the content clearly dictates otherwise. The term “and/or” means one or all of the listed elements or a combination of any two or more of the listed elements.

As used herein, “have”, “has”, “having”, “include”, “includes”, “including”, “comprise”, “comprises”, “comprising” or the like are used in their open-ended inclusive sense, and generally mean “include, but not limited to”, “includes, but not limited to”, or “including, but not limited to”.

“Optional” or “optionally” means that the subsequently described event, circumstance, or component, can or cannot occur, and that the description includes instances where the event, circumstance, or component, occurs and instances where it does not.

The words “preferred” and “preferably” refer to embodiments of the disclosure that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the inventive technology.

Ranges can be expressed herein as from “about” one particular value, and/or to “about” another particular value. When such a range is expressed, examples include from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

Also herein, the recitations of numerical ranges by endpoints include all numbers subsumed within that range (e.g., 1 to 5 includes 1, 1.5, 2, 2.75, 3, 3.80, 4, 5, etc.). It should be further understood that every numerical range given throughout this specification will include every narrower numerical range that falls within such broader numerical range, as if such narrower numerical ranges were all expressly written herein. Where a range of values is “greater than”, “less than”, etc. a particular value, that value is included within the range.

Any direction referred to herein, such as “top,” “bottom,” “left,” “right,” “upper,” “lower,” “above,” below,” and other directions and orientations are described herein for clarity in reference to the figures and are not to be limiting of an actual device or system or use of the device or system. Many of the devices, articles or systems described herein may be used in a number of directions and orientations. Directional descriptors used herein with regard to cell culture apparatuses often refer to directions when the apparatus is oriented for purposes of culturing cells in the apparatus.

It is also noted that recitations herein refer to a component being “configured” or “adapted to” function in a particular way. In this respect, such a component is “configured” or “adapted to” embody a particular property, or function in a particular manner, where such recitations are structural recitations as opposed to recitations of intended use. More specifically, the references herein to the manner in which a component is “configured” or “adapted to” denotes an existing physical condition of the component and, as such, is to be taken as a definite recitation of the structural characteristics of the component.

As used herein, the term "cell culture" refers to keeping cells alive in vitro. Included within this term are continuous cell lines (e.g., with an immortal phenotype), primary cell cultures, finite cell lines (e.g., non-transformed cells), and any other cell population maintained in vitro, including oocytes and embryos.

As used herein, the term “cell culture medium” refers to a nutrient source (in aspects, a liquid or a gel) used for growing or maintaining cells. As is understood by a person of skill in the art, the nutrient source may contain medium components required by the cell for growth and/or survival or may contain components that aid in cell growth and/or survival. Vitamins, essential and non-essential amino acids, proteins, carbohydrates, lipids, hormones, growth factors, minerals, serum, and trace elements are examples of medium components.

As used herein, the term “in vitro” refers to an artificial environment and to processes or reactions that occur within an artificial environment. In vitro environments can include, but are not limited to, test tubes and cell cultures. The term "in vivo" refers to the natural environment (e.g., an animal or a cell) and to processes or reaction that occur within a natural environment.

As used herein, “long-term culture” is meant to refer to cells (e.g., but not limited to hepatocytes) that have been cultured for at least about 12 hours, optionally for at least about 24 hours, for at least about 48 hours, or for at least about 72 hours, for at least about 96 hours, at least about 7 days, at least about 14 days, at least about 21 days, or at least about 28 days. Long-term culturing facilitates the establishment of functional properties, such as metabolic pathways, within the culture.

As used herein, the term “cell culture article” means any container useful for culturing cells and includes plates, wells, flasks, multi-well plates, multi-layer flasks, Transwell® inserts, Transwell® microcavity inserts, and perfusion systems which provide an environment for cell culture.

In aspects, a “well” is an individual cell culture environment provided in a multi-well plate format. In embodiments, a well can be a well of a 4 well plate, a 5 well plate, a 6 well plate, a 12 well plate, a 24 well plate, a 96 well plate, a 384 well plate, a 1536 well plate, or any other multi-well plate configuration.

As used herein chamber, wells, microwells, or microcavities “structured to constrain cells of interest to grow in 3D conformation” or the like means chambers, wells, microwells, or microcavities having dimensions or treatments, or a combination of dimensions and treatments, which encourage cells in culture to grow in 3D or spheroid conformation rather than as two-dimensional sheets of cells. Treatments include treatment with low binding solutions, treatments to render the surface less hydrophobic, or treatments for sterilization, for example.

As used herein “structured to provide” or “configured to provide” means that the article has features that provide the described result.

In aspects, a single “spheroid well” can be a well of a multi-well plate structured to constrain cells of interest to grow as a single 3D cell mass, or as a single spheroid (which may be differentiated to form an organoid), in that single spheroid well. For example, a well of a 96 well plate (wells of traditional 96 well plates) are approximately 10.67mm deep, have a top aperture of approximately 6.86mm and a well bottom diameter of approximately 6.35mm.

In aspects, “spheroid plate” means a multi-well plate having an array of single-spheroid wells.

In aspects, a well may have an array of “microcavities.” In embodiments, the “microcavity” can be, for example, a microwell that defines an upper aperture and a nadir, a center of the upper aperture, and a center axis between the nadir and the center of the upper aperture. In embodiments, the well is rotationally symmetrical about the axis (i.e. the sidewall is cylindrical). In some embodiments, the upper aperture defines a distance across the upper aperture of from between 250μm to 6mm, or any range within those measurements. In some embodiments the distance from the upper aperture to the nadir (the depth “d”) is between 200μm and 6mm, or between 400 and 600μm. The array of microcavities may have different geometries, for example, parabolic, hyperbolic, chevron, and cross-section geometries, or combinations thereof.

In aspects, a “microcavity spheroid plate” means a multi-well plate having an array of wells, each well having an array of microcavities.

In aspects, “round bottom” of a well or microcavity well can be, for example, a hemisphere, or a portion of a hemisphere, such as a horizontal section or slice of a hemisphere making up the bottom of the well or microcavity.

In aspects, the term “3D spheroid” or “spheroid” can be, for example, a ball of cells in culture, which are not a flat two-dimensional sheet of cells. The terms “3D spheroid” and “spheroid” are used interchangeably here. In aspects, the spheroid is comprised of a single cell type or multiple cell types, which may be differentiated to form an organoid, having a diameter of, for example, from about 100 microns to about 5mm, including intermediate values and ranges, depending on, for example, the types of cells in the spheroid or organoid. Spheroid diameters can be, for example, from about 100 to about 400 microns to avoid necrotic core formation. The maximum size of a spheroid is generally constrained to 400µm by diffusion considerations (for a review of spheroids and spheroid vessels see Achilli, T-M, et. al. Expert Opin. Biol. Ther. (2012) 12(10)).

As used herein “insert” means a cell culture well that fits into a well of a spheroid plate or a microcavity spheroid plate. The insert has sidewalls and a bottom surface defining a cavity for culturing cells. As used herein, a “Transwell® microcavity insert” means an insert in which the bottom surface has an array of microcavities.

As used herein “insert plate” means an insert plate containing an array of inserts structured to fit into an array of wells of a multi-well plate. As used herein, a “microcavity insert plate” means an insert plate in which each insert in the array of inserts has a bottom surface with an array of microcavities.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that any particular order be inferred. Any recited single or multiple feature or aspect in any one claim can be combined or permuted with any other recited feature or aspect in any other claim or claims.

While various features, elements or steps of particular embodiments may be disclosed using the transitional phrase “comprising,” it is to be understood that alternative embodiments, including those that may be described using the transitional phrases “consisting” or “consisting essentially of,” are implied.

As previously mentioned, cells cultured in three dimensions, such as spheroids or organoids, can exhibit more in vivo like functionality than their counterparts cultured in two dimensions as monolayers. Particularly, cells cultured in three dimensions more closely resemble in vivo tissue in terms of cellular communication and the development of extracellular matrices. Thus, there is an increasing demand for the storage and transportation of 3D cultured cells, such as spheroids and organoids, for various cell culture tests and examinations in numerous biotechnology-related fields. However, storage and transportation of 3D cultured cells, including spheroids and organoids, remains a challenge. In two-dimensional cell culture systems, cells can attach to a substrate on which they are cultured. However, often when cells are grown in three dimensions, such as spheroids and organoids, the cells interact with each other rather than attaching to the substrate, making such cells more susceptible to cell viability damage and integrity damage, and even cell death, due to disturbances during the storage and transportation process. Thus, it is difficult to transport stored cells cultured in three dimensions, such as spheroids and organoids.

The present disclosure describes, among other things, agarose and methylcellulose transport mediums, systems for cell storage and transport, and cell storage and transport methods for 3D cultured cells, including 3D spheroids and organoids. The present inventors unexpectedly found that the use of particular concentrations of a specific type of agarose in combination with particular concentration ranges of methylcellulose in cell culture medium provides for a cell storage and transport medium that is ideally suited for3D spheroid or organoid cell culture storage and transport. The instantly disclosed agarose and methylcellulose cell storage and transport medium is a firm gel at around 4° C, a soft gel at around 23° C, and a viscous liquid at 37° C. Thus, the cell storage and transport medium allows for 3D spheroid and organoid cells to be stored/transported/handled at a temperature range of between 4-37° C, which are optimal temperatures for maintaining cell viability and metabolic activity. This is important, for while the optimal temperature for the growth of many cell types is around 37° C, temperatures above this can result in negative effects to cell viability and integrity. Temperatures below this optimal temperature for cell growth, down to 4° C, result in decreased or slowed cell metabolism, but the cells are able to maintain their viability and integrity and return to normal growth activity when returned to normal or optimal growth conditions (e.g., around 37° C). Again, the instantly disclosed cell storage and transport medium is a firm gel at around 4° C, a soft gel at around 23° C, and a viscous liquid at 37° C. This allows for the transport of 3D spheroid or organoid cells at 4° C, as the cell storage and transport medium behaves like a solid gel, and thus provides sufficient strength to maintain cell viability and integrity during transport. Further, this solid gel state can be reversed to a liquid state at 37° C to permit 3D spheroid or organoid cell culture handling and recovery. Importantly, the use of the instantly-disclosed concentrations of a specific type of agarose in combination with particular concentration ranges of methylcellulose and cell culture medium allowed for the formation of a firm gel at around 4° C that does not resist gas transport through (e.g., oxygen and carbon dioxide transport through the gel), thus allowing for the improved ability to maintain 3D spheroid or organoid cell viability and integrity during transport at around 4° C. Surprisingly, the use of ultra-low gelling temperature agarose was required for the successful formation of a cell storage and transport medium with the above-described properties. In particular aspects, the transport mediums, systems and methods are used in combination with or performed in labware that combine 3D spheroid or organoid culture with a gas permeable, micro-patterned design that allows for protection and prolonged maintenance of spheroid cell (e.g. hepatocytes) or organoid viability and functionality during storage and transport, and also for multiple to several thousands of spheroids or organoids to be treated under the same conditions and medium, all while providing a physical barrier between individual 3D spheroids or organoids to prevent any spheroid fusion during culture or testing.

As such, in various embodiments, a cell storage and transport medium is disclosed. In aspects, the cell storage and transport medium comprises a mixture of cell culture medium, agarose, and methylcellulose, wherein the final agarose concentration in the storage and transport medium is about 0.5 to about 1.0% and the final methylcellulose concentration in the storage and transport medium is about 0.5 to about 0.7%.

Agarose is a thermally reversible gelling polysaccharide of alternating (1-3) linked β-D-galactose and (1-4) linked (3-6)-anhydrous-a-L-galactose copolymer that forms a gel at cold temperatures and can be melted at higher temperatures. Standard agarose becomes a gel at around 37° C, high gelling temperature agrarose becomes a gel at around 41° C, low gelling temperature agarose becomes a gel at 26-30° C, and ultra-low gelling temperature agarose becomes a gel at 8-17° C. The ultra-low gelling temperature agarose will re-melt at 55° C. Agarose is typically used to form gels used in molecular biology for separating strands of DNA based on mass, but it can also be used in viral plaque assays, and as a thickener. Agarose is supplied as a powder that must be added to water and melted at around 87° C to form an aqueous solution. Agarose has been used as a support for 3D cell culture. However, the use of agarose in transport mediums for culture systems would require the transported cells being subjected to temperatures of greater than 65° C to free them from the gel. It is well known that subjecting the cultured cells to such high temperatures can cause cell viability damage and even death to the cultured cells. Further, use of agaroses in transport mediums for culture systems often requires the use of agarase and additional steps for facilitating the removal of the agarose and thus allowing the release of the cells forming the transported culture.

Methylcellulose is a cellulose-derivative that is able to form a thermoreversible gel in aqueous media. When a methylcellulose-containing solution is heated, it will form a gel at the right concentration and temperature. Typically, a 2% solution of methylcellulose (w/w) has a gelation temperature of approximately 48° C. The gelation temperature drops linearly with increasing concentrations to about 30° C for a 10% solution. In the case of methylcellulose, it can go into solution in cold liquid, and after it is in solution as the solution is heated, it becomes a gel due to the intermolecular association of hydrophobic groups on the polymer chains. It is commonly used as a binder or thickener in pharmaceutical, cosmetic and food applications (1). It has also been used in the culture of cells when mixed with cell culture medium and is commonly used to enable formation of “embryoid bodies” from collections of stem cells since it keeps the cells in suspension due to its higher density than water or media without cellulose derivatives.

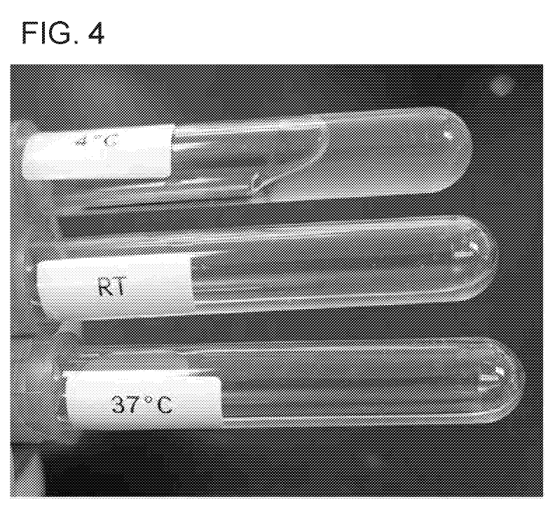

In some aspects of a cell storage and transport medium, the agarose is an ultra-low melting temperature agarose. In some aspects, the agarose has a gelling temperature of 8 - 17° C. Such ultra-low melting temperature agaroses and agaroses that have a gelling temperature of 8 - 17° C are commercially available and know in the art. The present inventors discovered that by combining ultra-low gelling temperature agarose, and methylcellulose the gelling properties of both components are modified to provide the ideal conditions for transporting cells as spheroids or organoids. The use of an ultra-low gelling temperature agarose (e.g., commercially available ultra-low gelling temperature agarose, such as but not limited to that sold by Sigma-Aldrich (Sigma product number A5030) in the instant cell storage and transport medium allows for the medium to form firm gel at around 4° C that provides sufficient strength to maintain 3D spheroid or organoid cell viability and integrity during transport, with the firm gel also allowing for gas transport through the gel (e.g., oxygen and carbon dioxide transport through the gel). This allows for the improved ability to maintain 3D spheroid and organoid cell viability and integrity during transport at around 4° C. In some aspects, the cell storage and transport medium is a firm gel at 4° C. In some aspects, the cell storage and transport medium is a soft gel at 23° C. In some aspects, the cell storage and transport medium is a viscous liquid at 37° C. FIG. 4 is a photograph showing the consistency of embodiments of the transportation medium at different temperatures. From the top of the image: at 4 °C, the consistency of the transport medium is a firm gel; at room temperature (RT, 23° C), consistency of the transport medium is a soft gel; and at 37° C, the transport medium is a viscous liquid. This is detectable in the images of FIG. 4 by the depth of the transport medium (pink material) in the tubes held at a slight angle which permits the gel to move toward the lower end of the tube.

In aspects, the cell storage and transport medium comprises a cell culture medium. A cell culture medium can include a natural medium or an artificial/synthetic medium (e.g., known cell culture media, including but not limited to: balanced salt solutions such as PBS, DPBS, HBSS, EBSS; basal media such as MEM or DMEM; or complex media such as RPMI-1640 or IMDM) along with additional natural products. In aspects, such artificial/synthetic medium can be serum containing, serum-free, chemically defined media, and/or protein-free media. In aspects, a cell culture medium can further include one or more of the following as is necessary to maintain the viability particular to the cell type being stored/transported, e.g., nutrients (one or more of, e.g., proteins, peptides, essential and/or nonessential amino acids), energy (one or more of, e.g., one or more of carbohydrates, such as glucose), essential metals and minerals (one or more of, e.g., calcium, magnesium, iron, phosphates, sulphates), buffering agents (one or more of, e.g., phosphates, acetates, bicarbonate), indicators for pH change (one or more of, e.g., phenol red, bromo-cresol purple), selective agents (one or more of, e.g., chemicals, antimicrobial agents), other basic supplements and growth factors known in the art (one or more of, sodium pyruvate, sodium bicarbonate, insulin, transferrin, selenium, and β-mercaptoethanol), etc. as are known in the art.

In various embodiments, a cell storage and transportation system that incorporates the above-described cell storage and transport medium (including all aspects thereof) is disclosed. The cell storage and transportation system includes labware that combine 3D spheroid culture with a gas permeable, micro-patterned design that allows for protection and prolonged maintenance of spheroid cell (e.g. hepatocytes) viability and functionality during storage and transport, and also for multiple to several hundreds of spheroids to be treated under the same conditions and medium, all while providing a physical barrier between individual 3D spheroids to prevent any spheroid fusion during culture or testing.

In aspects, the cell storage and transportation system comprises: cells; a cell culture article, wherein the cell culture article comprises a chamber, the chamber comprising an array of microcavities, each microcavity structured to constrain the cells to grow in a 3D spheroid or organoid confirmation; and a cell storage and transport medium comprising a mixture of cell culture medium, agarose, and methylcellulose, wherein the final agarose concentration in the storage and transport medium is about 0.5 to about 1.0% and the final methylcellulose concentration in the storage and transport medium is about 0.5 to about 0.7%.

In aspects of a cell storage and transportation system, the agarose of the cell storage and transport medium is an ultra-low gelling temperature agarose. In some aspects of a cell storage and transportation system, the agarose has a gelling temperature of 8 - 17° C. In some aspects, the cell storage and transport medium is a firm gel at 4° C. In some aspects of a cell storage and transportation system, the cell storage and transport medium is a soft gel at 23° C. In some aspects of a cell storage and transportation system, the cell storage and transport medium is a viscous liquid at 37° C.

In aspects of a cell storage and transportation system, the cell storage and transport medium comprises a cell culture medium. A cell culture medium can include a natural medium or an artificial/synthetic medium (e.g., known cell culture media, including but not limited to: balanced salt solutions such as PBS, DPBS, HBSS, EBSS; basal media such as MEM or DMEM; or complex media such as RPMI-1640 or IMDM) along with additional natural products. In aspects, such artificial/synthetic medium can be serum containing, serum-free, chemically defined media, and/or protein-free media. In aspects, a cell culture medium can further include one or more of the following as is necessary to maintain the viability particular to the cell type being stored/transported, e.g., nutrients (one or more of, e.g., proteins, peptides, essential and/or nonessential amino acids), energy (one or more of, e.g., one or more of carbohydrates, such as glucose), essential metals and minerals (one or more of, e.g., calcium, magnesium, iron, phosphates, sulphates), buffering agents (one or more of, e.g., phosphates, acetates, bicarbonate), indicators for pH change (one or more of, e.g., phenol red, bromo-cresol purple), selective agents (one or more of, e.g., chemicals, antimicrobial agents), other basic supplements and growth factors known in the art (one or more of, sodium pyruvate, sodium bicarbonate, insulin, transferrin, selenium, and β-mercaptoethanol), etc. as are known in the art.

In aspects of a cell storage and transportation system, each microcavity of the chamber comprises a top aperture and a liquid impermeable bottom comprising a bottom surface, wherein at least a portion of the bottom surface comprises a low-adhesion or no-adhesion material in or on the bottom surface. In some aspects, the liquid impermeable bottom including the bottom surface is gas-permeable. In some aspects, at least a portion of the bottom is transparent. In some embodiments, the bottom surface comprises a concave bottom surface. In some aspects, the chamber further comprises a side wall. In some aspects, the at least one concave bottom surface of each microcavity of the chamber includes a hemi-spherical surface, a conical surface having a taper of 30 to about 60 degrees from the side walls to the bottom surface, or a combination thereof. In some aspects, the cell culture article includes from 1 to about 2,000 of said chambers, wherein each chamber is physically separated from any other chamber. In some aspects, each chamber includes from about 1-to about 800 of said microcavities per square centimeter. For example, and not by way of limitation, a 6-well plate may comprise approximately 700 microcavities in one well of the 6-well plate, thus allowing for 700 spheroids (approximately 1.2 million cells in total) within one well. Similarly, a 24-well plate may comprise approximately 100-200 microcavities per well, while a 96-well plate may comprise approximately 50 microcavities per well. All of these numbers of microcavities are representative and based on the size of the microcavities.

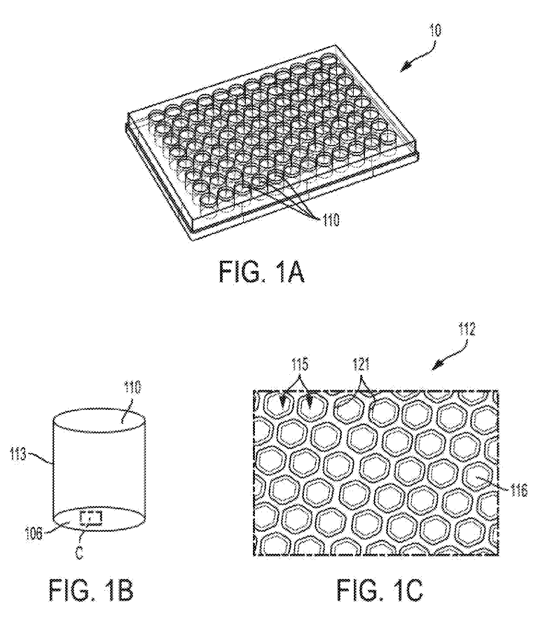

Referring now to FIGS. 1A, 1B and 1C, an exemplary cell culture article for use in aspects of a cell storage and transportation system and below methods is shown. The cell culture article shown is a microcavity spheroid plate, in this case a 96-well microcavity spheroid plate, having an array of microcavities on the bottom surface of each well, with each microcavity structured to constrain the cultured cells to grow in a 3D spheroid confirmation, to provide multiple spheroids in each of the 96 wells is shown. FIG. 1A illustrates a multi-well plate 10 having an array of wells 110. FIG. 1B illustrates a single well 110 of the multi-well plate 10 of FIG. 1A. The single well 110 has a top aperture, a liquid impermeable bottom surface 106, and a sidewall 113. FIG. 1C is an exploded view of the area of the bottom surface 106 of the well 110 shown in the box C in FIG. 1B illustrating an array of microcavities 112 in the bottom surface of the single well shown in FIG. 1B. Each microcavity 115 in the array of microcavities 112 has a sidewall 121 and a liquid impermeable bottom surface 116. The microcavity spheroid plate shown in FIGS. 1A, 1B and 1C, which provides an array of microcavities 112 in the bottom of each individual well 110, can be used to grow an individual 3D spheroid in each of the microcavities of each individual well of the multi-well plate. By using this type of vessel, a user can grow a large number of spheroids in each well of a multi-well plate and thereby provide a large number of 3D spheroids that maintain prolonged cell viability and functionality and that can be treated under the same culture and experimental conditions for use in various cultures or testing. Further, this type of vessel provides a physical barrier between individual 3D spheroids to prevent any spheroid fusion during culture or testing. Fusion of the spheroids can lead to the size of the fused spheroid surpassing the diffusion limits such that the cells in the core of the spheroid will start to lose viability or die. Thus, the microwell design of the instant disclosure provides a physical barrier that allows for the integrity of each spheroid to be maintained during extended incubation time.

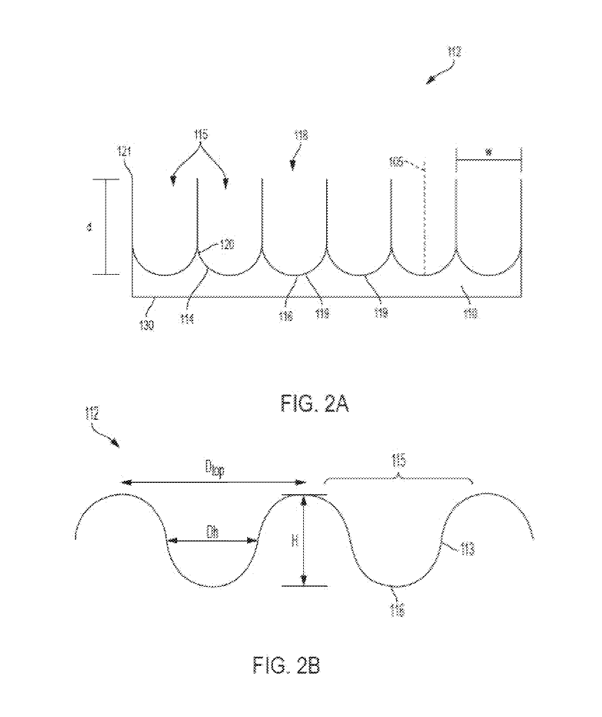

Referring now to FIG. 2A, an exemplary illustration of an array of microcavities 112 for use in aspects of a cell storage and transportation system and below methods is shown. FIG. 2A illustrates microcavities 115, each having top aperture 118, a bottom surface 119, a depth d, and a width w defined by sidewalls 121. As shown in FIG. 2A, the array of microcavities have a liquid impermeable, concave arcuate bottom surface 116. In embodiments, the bottom surfaces of the microcavities can be round or conical, angled, flat bottomed, or any shape suitable for forming 3D spheroids. A rounded bottom is preferred. The round bottom 119 can have a transition zone 114 as the perpendicular sidewalls transition into a round bottom 119. This can be a smooth or angled transition zone. In embodiments, the “microcavity” can be, for example, a microwell 115 that defines an upper aperture 118 and a nadir 116, a center of the upper aperture, and a center axis 105 between the nadir and the center of the upper aperture. In embodiments, the well is rotationally symmetrical about the axis (i.e. the sidewall is cylindrical). In some embodiments, the upper aperture defines a distance across the upper aperture (width w) of from between 250μm to 6mm, or any range within those measurements. In some embodiments the distance from the upper aperture to the nadir (the depth “d”) is between 200μm and 6mm, or between 400 and 600μm. The array of microcavities may have different geometries, for example, parabolic, hyperbolic, chevron, and cross-section geometries, or combinations thereof. In embodiments, the microcavities may have a protective layer 130 below them to protect them from direct contact with a surface such as a lab bench or a table. In some embodiments, there may be an air space 110 provided between the bottom of the wells 119 and the protective layer. In embodiments, the air space 110 may be in communication with the external environment or may be closed. Referring now to FIG. 2B, a further exemplary illustration of an array of microcavities 112 for use in aspects of a cell storage and transportation system and below methods is shown. FIG. 2B illustrates that the array of microcavities 112 may have a sinusoidal or parabolic shape. This shape creates a rounded top edge or microcavity edge which, in aspects, reduces the entrapment of air at a sharp corner or 90-degree angle at the top of a microcavity. As shown in FIG. 2B, in aspects, the microcavity 115 has a top opening having a top diameter Dtop, a height from the bottom of the microcavity 116 to the top of the microcavity H, a diameter of the microcavity at a height half-way between the top of the microcavity and the bottom 116 of the microcavity Dh, and a sidewall 113. In such aspects, the bottom of the well is rounded (e.g., hemispherically round), the side walls increase in diameter from the bottom of the well to the top and the boundary between wells is rounded. As such the top of the wells does not terminate at a right angle. In some aspects, a well has a diameter D at the half-way point (also termed Dh) between the bottom and top, a diameter Dtop at the top of the well and a height H from bottom to top of the well. In these embodiments, Dtop is greater than D.

In aspects of a cell storage and transportation system, the bottom surface of a microcavity having the at least one concave arcuate bottom surface or “cup” can be, for example, a hemi-spherical surface, a conical surface having a rounded bottom, and like surface geometries, or a combination thereof. The microcavity bottom ultimately terminates, ends, or bottoms-out in a spheroid “friendly” rounded or curved surface, such as a dimple, a pit, and like concave frustoconical relief surfaces, or combinations thereof. In aspects, the at least one concave surface of each microcavity in the chamber includes a hemi-spherical surface, a conical surface having a taper of 30 to about 60 degrees from the side walls to the bottom surface, or a combination thereof. In some aspects, the at least one concave arcuate bottom surface can be, for example, a portion of a hemisphere, such as a horizontal section or slice of a hemisphere, having a diameter of, for example, from about 250 to about 6,000 microns (i.e., 0.010 to 0.200 inch), including intermediate values and ranges, depending on, for example, the well geometry selected, the number of concave arcuate surfaces within each well, the number of wells in a plate, and like considerations. Other concave arcuate surface can have, for example, parabolic, hyperbolic, chevron, and like cross-section geometries, or combinations thereof.

In aspects of a cell storage and transportation system, the cell culture article comprising a chamber (e.g., a microcavity spheroid plate, a microcavity insert, a microcavity insert plate, etc.) can further comprise a low-adhesion, ultra-low adhesion, or no-adhesion coating on a portion of the chamber, such as on the at least one bottom surface or on the at least one concave bottom surface of each microcavity and/or one or more sidewalls. Examples of non-adherent material include polydimethylsiloxane, perfluorinated polymers, olefins, or like polymers, or mixtures thereof. Other examples include agarose, non-ionic hydrogels such as polyacrylamides, or polyethers such as polyethyleneoxide or polyols such as polyvinylalcohol, or like materials such as polyvinylpyrrolidone, or mixtures thereof.

In aspects of a cell storage and transportation system, the side wall surface (i.e., a surround) of the chamber and/or each microcavity can be, for example, a vertical cylinder or shaft, a portion of a vertical conic of decreasing diameter from the chamber top to the chamber bottom, a vertical square shaft or vertical oval shaft having a conical transition, i.e., a square or oval at the top of the well, transitioning to a conic, and ending with a bottom having at least one concave arcuate surface, i.e., rounded or curved, or a combination thereof. Other illustrative geometric examples include holey cylinders, holey conic cylinders, first cylinders then conics, and other like geometries, or combinations thereof.

In aspects of a cell storage and transportation system, one or more of, for example, a low-attachment substrate, the well curvature in the body and base portions of the microcavities, and gravity, can induce cells to self-assemble into spheroids or organoids. 3D spheroid or organoid cells maintain differentiated cell function indicative of a more in vivo-like, response relative to cells grown in a 2D monolayer. In aspects, the spheroid or organoid can be, for example, substantially a sphere, having a diameter of, for example, from about 100 microns to about 5 millimeters.

In aspects of a cell storage and transportation system, the cell culture article, including the chamber and/or each microcavity within the chamber, can further include opaque sidewalls and/or a gas permeable and liquid impermeable bottom comprising at least one concave surface. In some aspects, at least a portion of the bottom comprising at least one concave surface is transparent. Cell culture articles (e.g., a microcavity spheroid plate, a microcavity insert, a microcavity insert plate, etc.) having such features can provide several advantages for the instantly-disclosed methods, including removing the need for transferring the cultured cells from one multiwall plate (in which spheroids or organoids are formed and can be visualized) to another plate for conducting assays, therefore saving time and avoiding any unnecessary disruption of the spheroid. Further, a gas-permeable bottom (e.g., well-bottoms made from a polymer having a gas permeable property at a particular given thickness) can allow the 3D spheroid or organoid cells to receive increased oxygenation. An exemplary gas-permeable bottom can be formed from many types of polymers or polymer blends including polystyrene, polyolefins such as poly 4-methylpentane or polyethylene, polypropylene and their copolymers, polycarbonate, perfluorinated polymers or polymers such as polydimethylsiloxane at certain thicknesses. Representative thickness and ranges of gas permeable polymer can be, for example, from about 0.001 inch to about 0.025 inch, from 0.0015 inch to about 0.03 inch, including intermediate values and ranges dependent upon the permeability of oxygen and carbon dioxide of the specific polymer used (where 1 inch=25,400 microns; 0.000039 inches=1 micron). Additionally or alternatively, other materials having high gas permeability, such as polydimethylsiloxane polymers, can provide sufficient gas diffusion at a thickness, for example, of up to about 1 inch.

In aspects of a cell storage and transportation system, the cell culture article can further comprise a chamber annex, chamber extension area, or an auxiliary side chamber, for receiving a pipette tip for aspiration, the chamber annex or chamber extension (e.g., a side pocket) can be, for example, an integral surface adjacent to and in fluid communication with the chamber. The chamber annex can have a second bottom spaced away from the liquid impermeable bottom of the chamber and/or microcavities within the chamber. The chamber annex and the second bottom of the chamber annex can be, for example spaced away from the liquid impermeable bottom of the chamber such as at a higher elevation or relative altitude. The second bottom of the chamber annex deflects fluid dispensed from a pipette away from the liquid impermeable bottom of the chamber (and the liquid impermeable bottom of each microcavity within the chamber) to avoid disrupting or disturbing the spheroid.

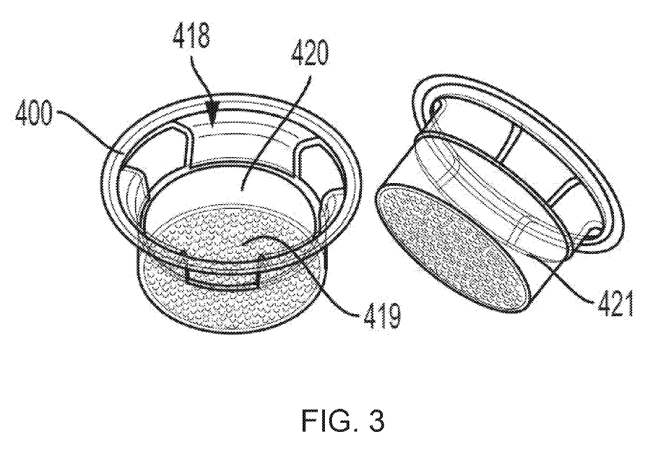

In aspects of a cell storage and transportation system, a microcavity insert or a microcavity insert plate can be used in combination with a cell culture article in the instantly disclosed systems and methods to culture the cells of interest to grow in 3D spheroid or organoid confirmation. For example, as shown in FIG. 3, an insert has a top aperture 418, sidewalls 421 and a bottom surface 419 forming an array of microcavities 420. It should be understood that inserts are available in many configurations, including but not limited to, 6 well microcavity inserts, 12 well microcavity inserts, 24 well microcavity inserts, 48 well microcavity inserts, 96 well microcavity inserts, as well as insert plate configurations where a single plate contains multiple inserts and the multi-well insert plate is structured to insert into the complimentary array of wells in a multi-well plate.

In various embodiments, a method for the transport of cells that incorporates the above-described cell storage and transportation system (including all aspects of the cell culture article thereof) and storage and transport medium (including all aspects disclosed above), is disclosed. In aspects, a method for the transport of cells includes a) culturing live cells in a cell culture article to form a spheroid, wherein the cell culture article comprises a chamber, the chamber comprising an array of microcavities, each microcavity structured to constrain the cells to grow in a 3D spheroid confirmation; b) adding a cell storage and transport medium comprising a mixture of agarose and methylcellulose and cell culture medium, wherein the final agarose concentration in the storage and transport medium is 0.5 to 1.0% and the final methylcellulose concentration in the storage and transport medium is 0.5 to 0.7% to the cell culture; c) solidifying the cell storage and transport medium; and d) transporting the cell culture article.

In aspects of a method for the transport of cells, the agarose of the cell storage and transport medium is an ultra-low gelling temperature agarose. In some aspects of a cell storage and transportation system, the agarose has a gelling temperature of 8-17° C. In some aspects, the cell storage and transport medium is a firm gel at 4° C. In some aspects of a cell storage and transportation system, the cell storage and transport medium is a soft gel at 23° C. In some aspects of a cell storage and transportation system, the cell storage and transport medium is a viscous liquid at 37° C.

In aspects of a method for the transport of cells, the cell storage and transport medium comprises a cell culture medium. A cell culture medium can include a natural medium or an artificial/synthetic medium (e.g., known cell culture media, including but not limited to: balanced salt solutions such as PBS, DPBS, HBSS, EBSS; basal media such as MEM or DMEM; or complex media such as RPMI-1640 or IMDM) along with additional natural products. In aspects, such artificial/synthetic medium can be serum containing, serum-free, chemically defined media, and/or protein-free media. In aspects, a cell culture medium can further include one or more of the following as is necessary to maintain the viability particular to the cell type being stored/transported, e.g., nutrients (one or more of, e.g., proteins, peptides, essential and/or nonessential amino acids), energy (one or more of, e.g., one or more of carbohydrates, such as glucose), essential metals and minerals (one or more of, e.g., calcium, magnesium, iron, phosphates, sulphates), buffering agents (one or more of, e.g., phosphates, acetates, bicarbonate), indicators for pH change (one or more of, e.g., phenol red, bromo-cresol purple), selective agents (one or more of, e.g., chemicals, antimicrobial agents), other basic supplements and growth factors known in the art (one or more of, sodium pyruvate, sodium bicarbonate, insulin, transferrin, selenium, and β-mercaptoethanol), etc. as are known in the art.

In aspects of a method for the transport of cells, each microcavity of the chamber comprises a top aperture and a liquid impermeable bottom comprising a bottom surface, wherein at least a portion of the bottom surface comprises a low-adhesion or no-adhesion material in or on the bottom surface. In some aspects, the liquid impermeable bottom including the bottom surface is gas-permeable. In some embodiments, at least a portion of the bottom is transparent. In some embodiments, the bottom surface comprises a concave bottom surface. In some aspects, the chamber further comprises a side wall. In some aspects, the at least one concave bottom surface of each microcavity of the chamber includes a hemi-spherical surface, a conical surface having a taper of 30 to about 60 degrees from the side walls to the bottom surface, or a combination thereof. In some aspects, the cell culture article includes from 1 to about 2,000 of said chambers, wherein each chamber is physically separated from any other chamber. In some embodiments, each chamber includes from about 1 to about 800 per of said microcavities square centimeter. For example, and not by way of limitation, a 6-well plate may comprise approximately 700 microcavities in one well of the 6-well plate, thus allowing for 700 spheroids (approximately 1.2 million cells in total) within one well. Similarly, a 24-well plate may comprise approximately 100-200 microcavities per well, while a 96-well plate may comprise approximately 50 microcavities per well. All of these numbers of microcavities are representative and based on the size of the microcavities.

In aspects, the cell culture article of the methods for the transport of cells includes the features disclosed in FIGS. 2-4.

In aspects of a method for the transport of cells, the bottom surface of a microcavity having the at least one concave arcuate bottom surface or “cup” can be, for example, a hemi-spherical surface, a conical surface having a rounded bottom, and like surface geometries, or a combination thereof. The microcavity bottom ultimately terminates, ends, or bottoms-out in a spheroid “friendly” rounded or curved surface, such as a dimple, a pit, and like concave frustoconical relief surfaces, or combinations thereof. In embodiments, the at least one concave surface of each microcavity in the chamber includes a hemi-spherical surface, a conical surface having a taper of 30 to about 60 degrees from the side walls to the bottom surface, or a combination thereof. In some embodiments, the at least one concave arcuate bottom surface can be, for example, a portion of a hemisphere, such as a horizontal section or slice of a hemisphere, having a diameter of, for example, from about 250 to about 5,000 microns (i.e., 0.010 to 0.200 inch), including intermediate values and ranges, depending on, for example, the well geometry selected, the number of concave arcuate surfaces within each well, the number of wells in a plate, and like considerations. Other concave arcuate surface can have, for example, parabolic, hyperbolic, chevron, and like cross-section geometries, or combinations thereof.

In aspects of a method for the transport of cells, the cell culture article comprising a chamber (e.g., a microcavity spheroid plate, a microcavity insert, a microcavity insert plate, etc.) can further comprise a low-adhesion, ultra-low adhesion, or no-adhesion coating on a portion of the chamber, such as on the at least one bottom surface or on the at least one concave bottom surface of each microcavity and/or one or more sidewalls. Examples of non-adherent material include polydimethylsiloxane, perfluorinated polymers, olefins, or like polymers, or mixtures thereof. Other examples include agarose, non-ionic hydrogels such as polyacrylamides, or polyethers such as polyethyleneoxide or polyols such as polyvinylalcohol, or like materials such as polyvinylpyrrolidone, or mixtures thereof.

In aspects of a method for the transport of cells, the side wall surface (i.e., a surround) of the chamber and/or each microcavity can be, for example, a vertical cylinder or shaft, a portion of a vertical conic of decreasing diameter from the chamber top to the chamber bottom, a vertical square shaft or vertical oval shaft having a conical transition, i.e., a square or oval at the top of the well, transitioning to a conic, and ending with a bottom having at least one concave arcuate surface, i.e., rounded or curved, or a combination thereof. Other illustrative geometric examples include holey cylinders, holey conic cylinders, first cylinders then conics, and other like geometries, or combinations thereof.

In aspects of a method for the transport of cells, one or more of, for example, a low-attachment substrate, the well curvature in the body and base portions of the microcavities, and gravity, can induce cells to self-assemble into spheroids. Cells grown in 3D maintain differentiated cell function indicative of a more in vivo-like, response relative to cells grown in a 2D monolayer. In embodiments, the spheroid or organoid can be, for example, substantially a sphere, having a diameter of, for example, from about 100 microns to about 5 millimeters.

In aspects of a method for the transport of cells, the cell culture article, including the chamber and/or each microcavity within the chamber, can further include opaque sidewalls and/or a gas permeable and liquid impermeable bottom comprising at least one concave surface. In some embodiments, at least a portion of the bottom comprising at least one concave surface is transparent. Cell culture articles (e.g., a microcavity spheroid plate, a microcavity insert, a microcavity insert plate, etc.) having such features can provide several advantages for the instantly-disclosed methods, including removing the need for transferring the cultured cells spheroid from one multiwall plate (in which spheroids are formed and can be visualized) to another plate for conducting assays, therefore saving time and avoiding any unnecessary disruption of the spheroid. Further, a gas-permeable bottom (e.g., well-bottoms made from a polymer having a gas permeable property at a particular given thickness) can allow the 3D spheroid or organoid to receive increased oxygenation. An exemplary gas-permeable bottom can be formed from many types of polymers or polymer blends including polystyrene, polyolefins such as poly 4-methylpentane or polyethylene, polypropylene and their copolymers, polycarbonate, perfluorinated polymers or polymers such as polydimethylsiloxane at certain thicknesses. Representative thickness and ranges of gas permeable polymer can be, for example, from about 0.001 inch to about 0.025 inch, from 0.0015 inch to about 0.03 inch, including intermediate values and ranges dependent upon the permeability of oxygen and carbon dioxide of the specific polymer used (where 1 inch=25,400 microns; 0.000039 inches=1 micron). Additionally or alternatively, other materials having high gas permeability, such as polydimethylsiloxane polymers, can provide sufficient gas diffusion at a thickness, for example, of up to about 1 inch.

In of a method for the transport of cells, the cell culture article can further comprise a chamber annex, chamber extension area, or an auxiliary side chamber, for receiving a pipette tip for aspiration, the chamber annex or chamber extension (e.g., a side pocket) can be, for example, an integral surface adjacent to and in fluid communication with the chamber. The chamber annex can have a second bottom spaced away from the liquid impermeable bottom of the chamber and/or microcavities within the chamber. The chamber annex and the second bottom of the chamber annex can be, for example spaced away from the liquid impermeable bottom of the chamber such as at a higher elevation or relative altitude. The second bottom of the chamber annex deflects fluid dispensed from a pipette away from the liquid impermeable bottom of the chamber (and the liquid impermeable bottom of each microcavity within the chamber) to avoid disrupting or disturbing the spheroid.

In aspects of a method for the transport of cells (as well as the instantly-disclosed cell storage and transportation system), the cells can be non-modified or genetically modified cells of any origin. Thus, cells can include animal cells, including but not limited to, human cells, rat cells, mouse cells, monkey cells, pig cells, dog cells, guinea pig cells, and fish cells. The cell can also include known established cell lines and primary animal cell cultures of pathological or nonpathological origin (including cancer cell lines). Such cells include, but not limited to, hepatocytes, renal cells, neurons, glial cells, non-glial cells, osteoblasts, osteocytes, osteoclasts, chondroblasts, chondrocytes, fibroblasts, keratinocytes, melanocytes, glandular cells, corneal cells, retinal cells, mesenchymal stem cells, hematopoietic stem cells, embryonic stem cells, induced pluripotent stem cells, epithelial cells, platelets, thymocytes, lymphocytes, monocytes, macrophages, myocytes, urethral cells and/or germ cells.

In aspects of a method for the transport of cells, a) comprises maintaining the cells in standard culture conditions for the particular cell type in culture until reaching b). In some embodiments of the instantly-disclosed methods, the cultured cells that form a 3D spheroid or organoid, are cultured as a long-term culture. In some embodiments, the cultured cells are cultured for at least about 12 hours, optionally for at least about 24 hours, for at least about 48 hours, or for at least about 72 hours, for at least about 96 hours, at least about 7 days, at least about 14 days, at least about 21 days, or at least about 28 days, or at least about3-4 months in the case of some organoids. Long-term culturing facilitates the establishment of functional properties, such as metabolic pathways, within the culture.

In aspects of a method for the transport of cells, b) comprises adding cell storage and transport medium to the cells in culture at 37° C. In aspects, any cell culture media that is used to culture the 3D spheroid cells or organoids is removed before the addition of the cell storage and transport medium in b).

In aspects of a method for the transport of cells, c) is carried out at a temperature of about 4° C so that the cell storage and transport medium is a firm gel. In aspects of a method for the transport of cells, d) transporting the culture article is carried out at a temperature of about 4° C so that the cell storage and transport medium is a firm gel and thus provides sufficient strength to maintain cell viability and integrity during transport.

In aspects of a method for the transport of cells, the transport time is not more than 48 hours or not more than 72 hours. In aspects, the transport time is between 0-48 hours or 0-72 hours, including any value or range therebetween. Transportation can be of any transportation method known in the art, including by truck, car, plane, boat, etc.

In aspects of a method of the transport of cells, the method further includes sealing the cell culture chamber before transport. For example, the open portion formed in the top aperture of the each microcavity of the chamber of the cell culture article may be covered by a seal means, such as a film, cap, or lid, to separate the cells from the outside during storage/transport. As is known in the art, the seal means may be made of a material that blocks or allows a flow of liquid or gas, such as blocking or allowing carbon dioxide or oxygen to permeate.

In aspects of a method of the transport of cells, the method further includes e) recovery of the transported cells so that they can be used in different applications, including further culturing and/or testing. In aspects, e) comprises removing the cell storage and transport medium and replacing it with culture medium. In aspects, e) includes incubating the cell culture article at about 37° C for at least about 1 hr; and subsequently removing the cell storage and transport medium and replacing it with culture medium. Thus, the solid gel state of the cell storage and transport medium can be reversed to a liquid state at 37° C to permit 3D spheroid or organoid cell culture handling and recovery. In aspects, e) includes adding cell culture media that is about 37° C to the cell storage and transport medium; incubating the cell culture article at about 37° C for at least about 1 hr; and removing the cell storage and transport medium and replacing it with culture medium. In aspects, e) includes incubating the cell culture article at about 37° C for at least about 1 hr; and subsequently removing the cell storage and transport medium and extracting the 3D spheroid or organoid cells from the cell culture article.

### ASPECTS

A variety of aspects of compositions, systems, and methods have been described herein. A summary of a few select examples of such compositions, systems, and methods are provided below.

A 1^{st} aspect is a cell storage and transport medium comprising a mixture of agarose and methylcellulose and cell culture medium, wherein the final agarose concentration in the storage and transport medium is about 0.5 to about 1.0% and the final methylcellulose concentration in the storage and transport medium is about 0.5 to about 0.7%.

A 2^{nd} aspect is a cell storage and transport medium of the 1st aspect, wherein the agarose is an ultra-low gelling temperature agarose.

A 3^{rd} aspect is a cell storage and transport medium of the 1st or 2nd aspect, wherein the agarose has a gelling temperature of 8-17° C.

A 4^{th} aspect is a cell storage and transport medium of any of aspects 1-3, wherein the cell storage and transport medium is a firm gel at 4° C.

A 5^{th} aspect is a cell storage and transport medium of aspects 1-4, wherein the cell storage and transport medium is a soft gel at 23° C.

A 6^{th} aspect is a cell storage and transport medium of any of aspects 1-5, wherein the cell storage and transport medium is a viscous liquid at 37° C.

A 7^{th} aspect is a cell storage and transportation system, said system comprising: cells; a cell culture article, wherein the cell culture article comprises a chamber, the chamber comprising an array of microcavities, each microcavity structured to constrain the cells to grow in a 3D spheroid confirmation; and a cell storage and transport medium comprising a mixture of cell culture medium, agarose and methylcellulose, wherein the final agarose concentration in the storage and transport medium is 0.5 to 1.0% and the final methylcellulose concentration in the storage and transport medium is about 0.5 to about 0.7%.

An 8^{th} aspect is a cell storage and transportation system of aspects 7, wherein each microcavity of the chamber comprises: a top aperture; and a liquid impermeable bottom comprising a bottom surface, wherein at least a portion of the bottom comprises a low-adhesion or no-adhesion material in or on the bottom surface.

A 9^{th} aspect is a cell storage and transportation system of aspect 8, wherein the liquid impermeable bottom comprising the bottom surface is gas permeable.

A 10^{th} aspect is a cell storage and transportation system of any of aspects 8-9, wherein the bottom surface comprises a concave bottom surface.

An 11^{th} aspect is a cell storage and transportation system of any of aspects 8-10, wherein at least a portion of the bottom is transparent.

A 12^{th} aspect is a cell storage and transportation system of aspect 10, wherein the concave surface comprises a hemi-spherical surface, a conical surface having a taper of 30 to about 60 degrees from the side walls to the bottom surface, or a combination thereof.

A 13^{th} aspect is a cell storage and transportation system of any of aspects 7-12, wherein each microcavity of the chamber further comprises a side wall.

A 14^{th} aspect is a cell storage and transportation system of aspect 13, wherein the side wall surface comprises a vertical cylinder, a portion of a vertical conic of decreasing diameter form the chamber’s top to bottom surface, a vertical square shaft having a conical transition to the concave bottom surface, or a combination thereof.

A 15^{th} aspect is a cell storage and transportation system of any of aspects 7-14, wherein the cell culture article comprises from 1 to about 2,000 of said chambers, wherein each chamber is physically separated from any other chamber.

A 16^{th} aspect is a cell storage and transportation system of any of aspects 7-15, wherein the agarose is an ultra-low gelling temperature agarose.

A 17^{th} aspect is a cell storage and transportation system of any of aspects 7-16, wherein the agarose has a gelling temperature of 8 - 17° C.

An 18^{th} aspect is a cell storage and transportation system of any of aspects 7-17, wherein the cell storage and transport medium is a firm gel at 4° C.

A 19th aspect is a cell storage and transportation system of any of aspects 7-18, wherein the cell storage and transport medium is a soft gel at 23° C.

A 20^{th} aspect is a cell storage and transportation system of any of aspects 7-19, wherein the cell storage and transport medium is a viscous liquid at 37° C.

A 21^{st} aspect is a method for the transport of cells comprising: a) culturing live cells in a cell culture article to form a spheroid or organoid, wherein the cell culture article comprises a chamber, the chamber comprising an array of microcavities, each microcavity structured to constrain the cells to grow in a 3D spheroid or organoid confirmation; b) adding a cell storage and transport medium comprising a mixture of cell culture medium, agarose and methylcellulose, wherein the final agarose concentration in the storage and transport medium is about 0.5 to about 1.0% and the final methylcellulose concentration in the storage and transport medium is about 0.5 to about 0.7% to the cell culture; c) solidifying the cell storage and transport medium; and d) transporting the cell culture article.

A 22^{nd} aspect is a method of aspect 21, further comprising wherein each microcavity of the chamber comprises: a top aperture; and a liquid impermeable bottom comprising a bottom surface, wherein at least a portion of the bottom comprises a low-adhesion or no-adhesion material in or on the bottom surface.

A 23^{rd} aspect is a method of aspect 22, wherein the liquid impermeable bottom comprising the bottom surface is gas permeable.

A 24^{th} aspect is a method of any of aspects 22-23, wherein the bottom surface comprises a concave bottom surface.

An 25^{th} aspect is a method of any of aspects 22-24, wherein at least a portion of the bottom is transparent.

A 26^{th} aspect is a method of any of aspects 22-25, wherein the concave surface comprises a hemi-spherical surface, a conical surface having a taper of 30 to about 60 degrees from the side walls to the bottom surface, or a combination thereof.

A 27^{th} aspect is a method of any of aspects 21-26, wherein each microcavity of the chamber further comprises a side wall.

A 28^{th} aspect is a method of aspect 27, wherein the side wall surface comprises a vertical cylinder, a portion of a vertical conic of decreasing diameter form the chamber’s top to bottom surface, a vertical square shaft having a conical transition to the concave bottom surface, or a combination thereof.

A 29^{th} aspect is a method of any of aspects 21-28, wherein the cell culture article comprises from 1 to about 2,000 of said chambers, wherein each chamber is physically separated from any other chamber.

A 30^{th} aspect is a method of any one of aspects 21-29, wherein the agarose is an ultra-low gelling temperature agarose.

A 31^{st} aspect is a method of any one of aspects 21-30, wherein the agarose has a gelling temperature of 8 - 17° C.

A 32^{nd} aspect is a method of any of aspects 21-31, wherein the cell storage and transport medium is a firm gel at 4° C.

A 33^{rd} aspects is a method of any one of aspects 21-32, wherein the cell storage and transport medium is a soft gel at 23° C.

A 34^{th} aspect is a method of any of aspects 21-33, wherein the cell storage and transport medium is a viscous liquid at 37° C.

A 35^{th} aspect is a method of any of aspects 21-34, wherein b) comprises adding cell storage and transport medium at 37° C to the 3D spheroid cells or organoids. In aspects, any cell culture media that is used to culture the 3D spheroid cells or organoids is removed before the addition of the cell storage and transport medium in step b).

A 36^{th} aspect is a method of any of aspects 21-35, wherein step c) is carried out at a temperature of about 4° C.

A 37^{th} aspect is a method of any of aspects 21-36, wherein step d) is carried out at a temperature of about 4° C.

A 38^{th} aspect is a method of any of aspects 21-37, wherein the transport time is not more than 48 or 72 hours.

A 39^{th} aspect is a method of any of aspects 21-38, further comprising sealing the cell culture chamber.

A 40^{th} aspect is a method of any of aspects 21-39, further comprising: e) recovery of the transported cells.

A 41^{st} aspect is a method of aspect 40, wherein e) comprises removing the transport medium and replacing it with culture medium.

A 42^{nd} aspect is a method of aspect 40, wherein e) comprises incubating the cell culture article at about 37° C for at least about 1 hr; and subsequently removing the transport medium and replacing it with culture medium.

A 43^{rd} aspect is a method of aspect 40, wherein e) comprises adding cell culture media that is about 37° C to the transport medium; incubating the cell culture article at about 37° C for at least about 1 hr; and removing the transport medium and replacing it with culture medium.

A 44^{th} aspect is a method of aspect 40, wherein e) comprises incubating the cell culture article at about 37° C for at least about 1 hr; and subsequently removing the transport medium and extracting the 3D spheroid cells from the cell culture article.

### EXAMPLES

The following examples serve to illustrate certain preferred embodiments and aspects of the present disclosure and are not to be construed as limiting the scope thereof.

### Example 1

### Cell Storage and Transport Medium Formulations

Previous cell storage and transport medium evaluations demonstrated that the maximum and minimum concentrations for the ultra-low gelling agarose solution are 1.0% and 0.5% and the maximum for the methylcellulose solution is 1%. The mixture also exhibits some properties of a non-Newtonian fluid, in that if the mixture seems to be solid at 37 degrees C, exerting a shear force on the mixture (by tapping the container), will cause the mixture to liquefy. This property is commonly referred to as “shear-thinning”. Here, concentration ranges for ultra-low gelling agarose and methylcellulose are further evaluated.

### Materials

Initial evaluation: Ultra-low Gelling Temperature Agarose (AGR-L, Sigma Cat. No. A5030), prepared to 4% in cell culture grade water and steam sterilized; Methylcellulose Concentrate (Mc, R&D systems Cat. No. HSC011) 2.8% (in water, sterile); 2x IMDM (from 4x stock), supplemented with; 20% FBS (fetal bovine serum), 4 mM Glutagro™, 6.04 g/L Sodium Bicarb. (NaHCO3) and 2x ITS (insulin, transferrin, selenium) solution; 1x IMDM, without supplements; Cell culture grade water; and - ULA (UltraLow Attachment) coated 60 mm dishes

Cell culture evaluation was conducted with the following: HT-29 spheroid cultures generated in T25 Microcavity vessels; Growth medium was McCoy’s with 10% FBS; spheroid cultures were observed with an Evos-FL microscope; TrypLE and Trypsin/EDTA were used as dissociation regents; and a NucleoCounter® was used for cell innumeration and viability assessment.

### Performance/Evaluation After Cold Storage

The cell storage and transport medium samples were evaluated at room temperature to monitor consistency of solutions as storage temperature changed from 4° C to room temperature.

### Methods

Initial evaluation without cell culture:

1. Ultra-low gelling temperature agarose (AGR-L) solutions:
a. 2% AGR-L (12mL), 1:1 dilution of 4% AGR-L and 2x IMDM
b. 1% AGR-L (8mL), 1:1 dilution of 2% AGR-L and 1x IMDM

2. Methylcellulose (Mc) solutions:
a. 2% Mc (8mL), dilute 2.8% stock with 2x IMDM
b. 1.4% Mc (5mL), 1:1 dilution of 2.8% stock with 2x IMDM
c. 1% Mc (5mL), 1:1 dilution of 2% with 1x IMDM
d. 0.7% Mc (5mL), 1:1 dilution of 1.4% with 1x IMDM

3. Cell storage and transport medium formulations with 1% AGR-L 4ml per sample:
a. 1% AGR-L & 1% Mc - 1:1 dilution of 2% AGR-L and 2% Mc
b. 1% AGR-L & 0.7% Mc - 1:1 dilution of 2% AGR-L and 1.4% Mc
c. 1% AGR-L & 0.5% Mc - 1:1 dilution of 2% AGR-L and 1% Mc
d. 1% AGR-L & 0.35% Mc - 1:1 dilution of 2% AGR-L and 0.7% Mc

4. Cell storage and transport medium formulations with 0.5% AGR-L, 4mL per sample:
a. 0.5% AGR-L & 1% Mc - 1:1 dilution of 2% AGR-L and 2% Mc
b. 0.5% AGR-L & 0.7% Mc - 1:1 dilution of 2% AGR-L and 1.4% Mc
c. 0.5% AGR-L & 0.5% Mc - 1:1 dilution of 2% AGR-L and 1% Mc
d. 0.5% AGR-L & 0.35% Mc - 1:1 dilution of 2% AGR-L and 0.7% Mc

5. Dispensed TM solutions into - ULA -coated 60mm dishes

6. Incubated dishes at 37° C for 20 minutes then transferred to 4° C storage overnight

7. Evaluated media formulations for the following properties after cold storage;
a. Ability of cell storage and transport mediums to solidify at cold temperature and remain solid as it reaches room temperature
b. Ability of cell storage and transport mediums change phase (solid to liquid) by tapping

8. Evaluated ease of cell storage and transport medium removal from culture (dishes)
a. To dilute cell storage and transport mediums, added 2mL of PBS per dish, incubated dishes at 37 ˚C for 30 minutes
b. Removed/ aspirated cell storage and transport medium from dishes
c. Repeated dilution/ incubation cycles a total of three times

### Notes/Observations

The formulation at 1% concentration for both AGR-L and Mc were too thick and difficult to work with.

Cell storage and transport medium formulated with Mc concentrations at lower than 0.7% were very easy to work with; easy to mix, dispense and spread out evenly in dish.

After incubation at 37° C, all but the 1% cell storage and transport medium formulations changed phase (liquified) and spread out evenly in the dish.

0.5% AGR-L/0.35% combination is too thin.

### Results

1% AGR-L TM formulations after cold storage

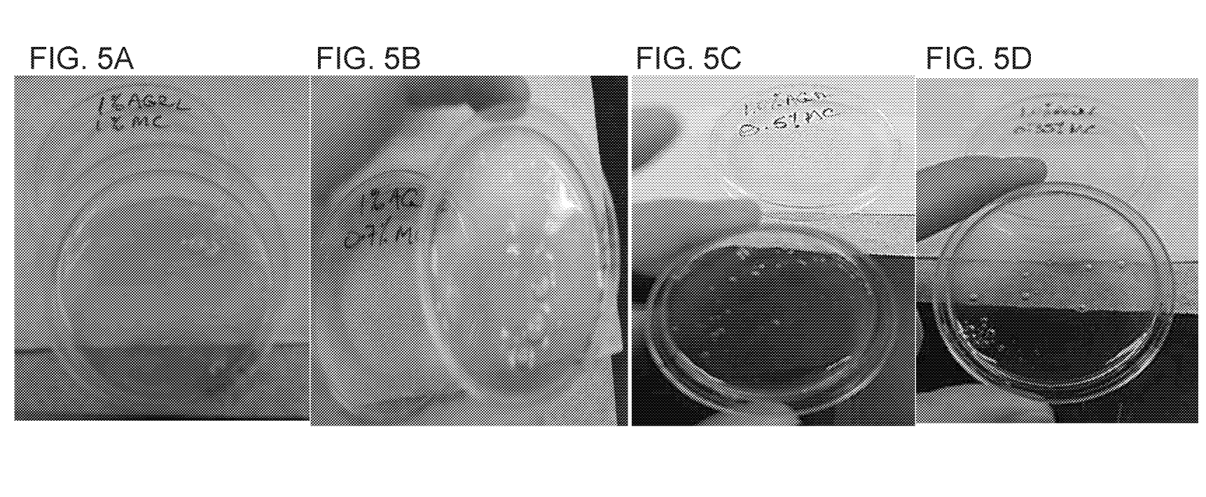

1% AGR-L/ 1% Mc – Is a thick solution and solid at cold temperature. The solution did not change phase (liquify) with tapping. FIG. 5A shows the formulation as a solid plug sliding off the surface as the dish is tipped forward.

1% AGR-L/0.7% Mc – I a thick solution and solid at cold temperature with some liquification of material after tapping. Material softened quickly into a viscous gel like material at room temperature. FIG. 5B shows the formulation as a thin covering of the thick/viscous material remaining on the surface as the dish is tipped forward.

1 % AGR-L/ 0.5 % Mc –This formulation is very similar to formulation with 0.7% Mc, which is easier to mix during preparation. Although solid at cold temperature, it quickly softened into a viscous gel like material at room temperature with liquification after tapping. FIG. 5C shows the formulation as a viscous material remaining on the surface as the dish is tipped forward.

1 % AGR-L/ 0.35 % Mc – This formulation did not solidify, but rather it formed into a soft material at 4° C that quickly liquefied out of cold storage. FIG. 5D shows the formulation liquid pooling at the bottom as dish is slightly tipped forward.

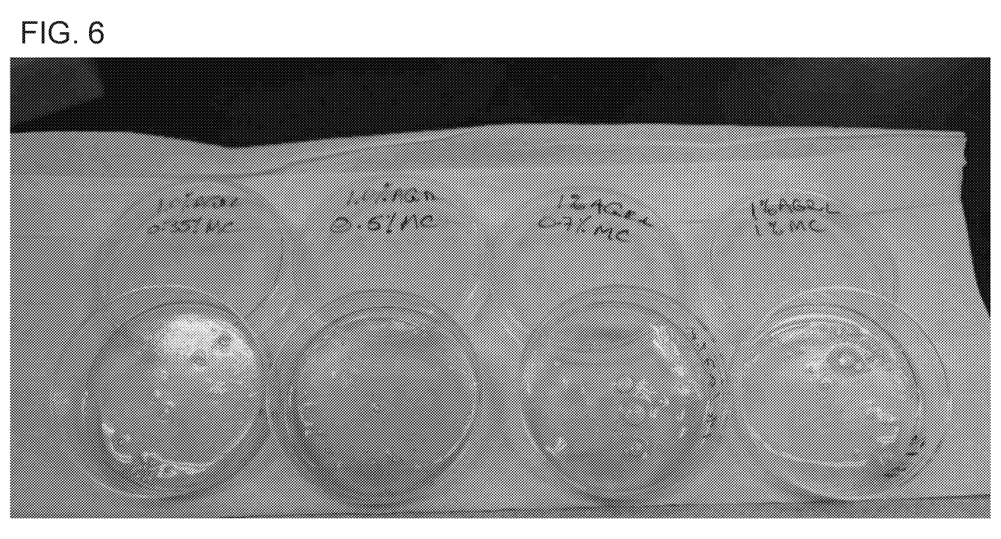

1 % AGR-L cell storage and transport medium formulations after cold storage and after being allowed to reach room temperature (23° C). Images of 1% AGR-L cell storage and transport medium formulations in ULA-coated 60mm dishes after being allowed to reach room temperature are shown in FIG. 6. FIG. 6 shows, from left to right, 1% AGR-L/0.35% Mc, 1% AGR-L/0. 5% Mc, 1% AGR-L/0.7% Mc, and 1% AGR-L/1.% Mc. Dishes were tipped forward to demonstrate consistency of cell storage and transport medium formulations at room temperature. The components of the 0.35% Mc formulation separated at room temperature. Image shows the liquid component pooling at the bottom of the dish while the thicker components remain on the surface of the dish. Both the 0.5% to 0.7% Mc concentrations quickly softened into a viscous gel like material but showed retention to the surface even while tipped forward. There was slight separation of liquid media from other components was observed for the 0.5% concentration. Image shows a small pool of liquid (media) at bottom of dish while viscous materials remain in place. The 1% Mc formulation remained solid with some liquid separation.

0.5% AGR-L cell storage and transport medium formulations after cold storage

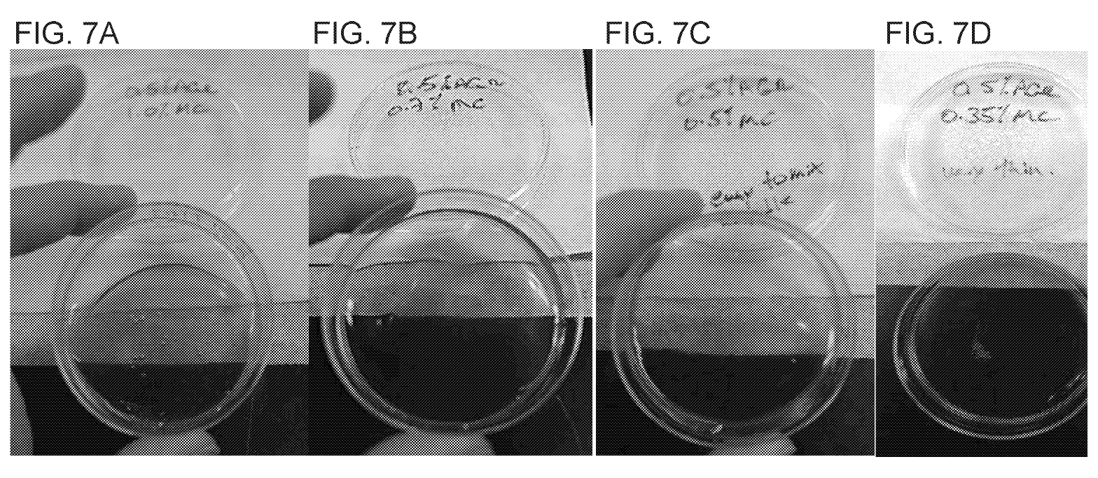

0.5% AGR-L/1% Mc – This is a thick solution but easy to work with. It formed a solid plug at 4° C. FIG. 7A shows the formulation plug sliding down as dish is tipped forward. Material did soften slightly after tapping.

0.5% AGR-L/0.7% Mc –Is very similar to above formulation for mixing and preparation. Solid at cold temperature, tapping increased a change of phase from solid to a viscous gel. FIG. 7B shows the formulation viscous material maintaining its place as dish is tipped forward.

0.5% AGR-L/0.5% Mc –This was easy to work with during the mixing steps. It formed into a thick gel like material at 4° C. FIG. 7C shows the formulation gel like material sliding down as dish is tipped forward.

0.5% AGR-L/0.35% Mc – This was easy to work with during the mixing steps. As shown in FIG. 7D, the formulation did not solidify at 4° C but formed a gel like material that quickly turned into a viscous liquid once removed from cold storage.

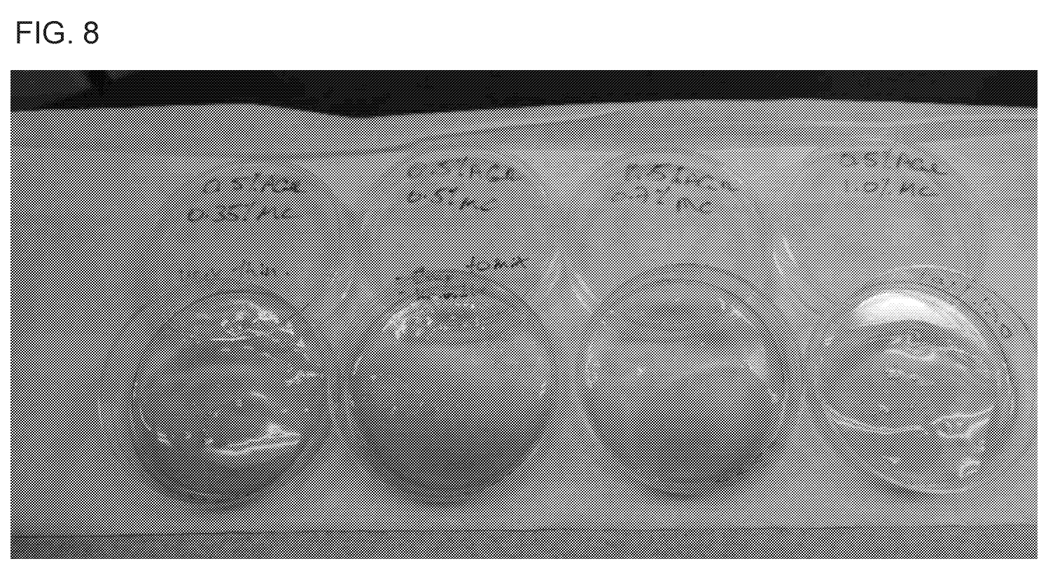

0.5% AGR-L cell storage and transport medium formulations after cold storage and after being allowed to reach room temperature. Images of 0.5% AGR-L cell storage and transport medium formulations in ULA-coated 60mm dishes after being allowed to reach room temperature are shown in FIG. 8. FIG. 8 shows, from left to right, 0.5% AGR-L/0.35% Mc, 0.5% AGR-L/0. 5% Mc, 0.5% AGR-L/0.7% Mc, and 0.5% AGR-L/1.0% Mc. Dishes were tipped forward to demonstrate the consistency of the formulations at room temp. The 0.35% Mc formulation became a thick liquid at room temperature with visible separation of the cell storage and transport medium formulations components. Both the 0.7% and 0.5 %Mc formulations quickly turned into a viscous gel at room temperature with some separation of components (AGR-L and Mc) after tapping. The 1.0% Mc formulation softened into a gel like plug at room temperature.

### Notes/Observations

The 1.0% AGR-L formulations maintain thicker/more solid like consistency at room temperature.

The 0.5% AGR-L formulations quickly turn into soft gels at room temperature.

Lower combinations of AGR-L and Mc appear unstable, as components fall out of solution after cold storage.

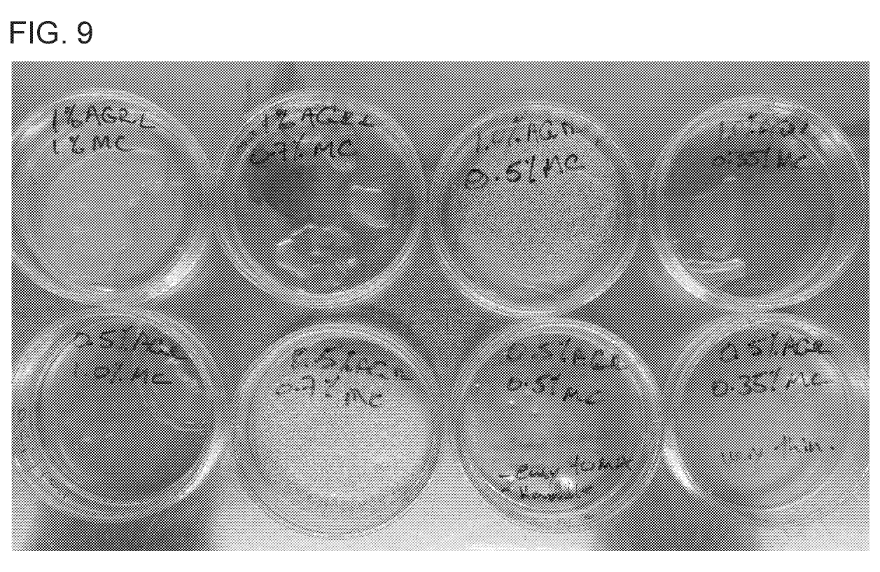

Cell storage and transport medium formulation removal. To evaluate ease of removal of cell storage and transport medium formulations, PBS was added to samples (2mL per dish). Samples were then incubated at 37° C for up to 30 minutes. Diluted/ softened cell storage and transport medium formulation was aspirated out of the dish. The dilution/ heat cycle was repeated a total of three times. Results are shown in FIG. 9. FIG. 9 shows images of ULA-coated 60 mm dishes with residual cell storage and transport medium formulation material after three dilution/ heat cycles and removal of liquefied/softened material. FIG. 9 shows dishes with covers for identification purposes, In FIG. 9, the samples on the top row, from left to right include 1.0% AGR-L/1% Mc, 1.0% AGR-L/0.7% Mc, 1.0% AGR-L/0.5% Mc, and 1.0% AGR-L/0.35% Mc, while the samples on the bottom row, from left to right, include 0.5% AGR-L/1.0% Mc, 0.5% AGR-L/0.7% Mc, 0.5% AGR-L/0.5% Mc, and 0.5% AGR-L/0.35% Mc.

### Notes/Observations

For all the 1% AGR-L combinations (top row of FIGS. 9), solid chunks of cell storage and transport medium formulations remain in dishes after dilution/aspiration steps.

Cell storage and transport mediums formulated with 1% Mc were also difficult to dissolve/dilute out.

At 0.5% AGR-L (bottom row of FIGS. 9) the majority of cell storage and transport medium formulations were easily removed from dishes, especially when working at the lower Mc concentrations (0.5% to .35%).

At 0.35% Mc some separation/ clumping of materials was observed.

### Summary

The 0.5 % AGR-L formulations maintained thick viscous like consistency at cold storage but softened quickly at room temperature. They were harder to work with at warmer temperatures but easier to remove from the culture when compared to the 1.0% AGR-L formulations.

The 1.0 % Mc concentration was too difficult to work with and it did not generate the desired viscosity for easy removal.

0.35% Mc concentration appears to be too low to maintain the desired consistency of cell storage and transport medium formulations.

The 0.5% AGR-L formulations quickly turn into soft gels at room temperature.

Lower combinations of AGR-L and Mc appear unstable, as components fall out of solution after cold storage.

### Cell culture evaluation

The cell storage and transport medium samples were evaluated for cell culture, particularly 0.5% AGR-L concentration with Mc concentration ranges from 0.7% to 0.35% as compared to a 1.0% AGR-L/0.7% Mc formulation.

### Methods

1. Generated HT-29 spheroids (48 hr. cultures) in T25 Microcavity flasks

2. Prepared cell storage and transport medium formulations for evaluation;
a. 1% AGR-L/0.7% Mc (control)
b. 0.5% AGR-L and Mc at 0.7%, 0.5% and 0.35% concentrations

3. Removed spent medium from vessel, replaced with 4mL of cell storage and transport medium formulations. One vessel per condition.

4. Incubated flasks at 37 ˚C for 30 minutes to allow even dispersal of cell storage and transport medium formulations.

5. Packaged flasks in Styrofoam box with ice packs to mimic shipping conditions. Stored box overnight at 4° C.

6. Ship/drop test; removed box from cold storage, flip and drop box four times to mimic shipping conditions.

7. Evaluated cultures for; health of spheroids, spheroid retention, ease of cell storage and transport medium formulations removal from cell cultures.
a. Monitored cell health by tracking growth of spheroid cultures
b. Monitored spheroid retention by imaging cultures before and after evaluation
c. Evaluated cell storage and transport medium formulation removal using three dilution/heat cycles to remove cell storage and transport medium formulations from vessels.

### Notes/Observations

0.5% AGR-L/0.5% Mc concentrations easiest to mix and work with.

0.5% and 0.35% Mc cell storage and transport medium formulations are too thin, some dislodging of spheroids during initial addition of cell storage and transport medium formulations to flasks.

Under cold conditions (4° C), spheroids remained in place after ship/drop test for all cell storage and transport medium formulations.

After cell storage and transport medium formulations evaluation, no noticeable negative affects to spheroid cultures, all appear similar to control.

### Results of Cell Storage and Transport Medium Formulations Removal and Culture Recovery

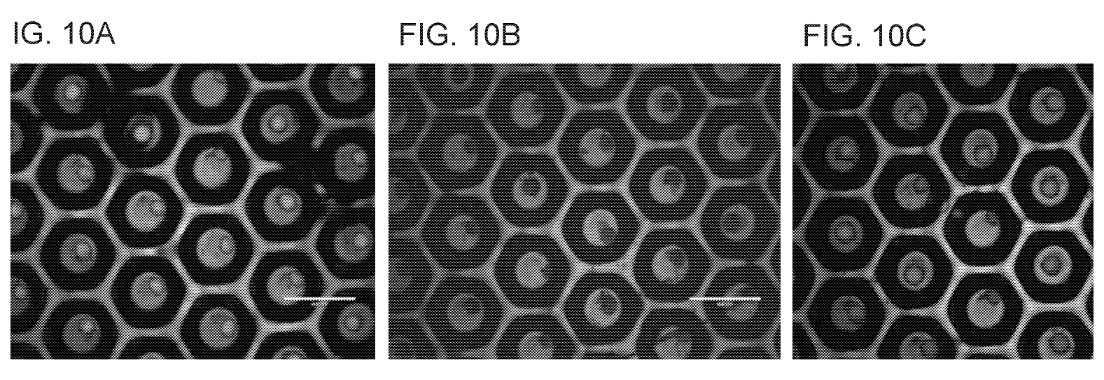

Results are shown in FIGS. 10A-C, 11A-C, 12A-C, 13A-C, and 14.

FIGS. 10A-C show images of HT-29 spheroid cultures (at 2x magnification) for the 1% AGR-L/0.7% Mc formulation (control). FIG. 10A shows images of spheroid cultures after 4° C storage during which the spheroids remained in place. FIG. 10B shows images after three dilution cycles where about 20% of TM remained in vessel in the form of a gel like material covering the surface. After cell storage and transport medium removal, spheroids appear somewhat irregular. FIG. 10C shows an image of the HT29 spheroid cultures 24 hr after removal of cell storage and transport medium that are in a recovery period.

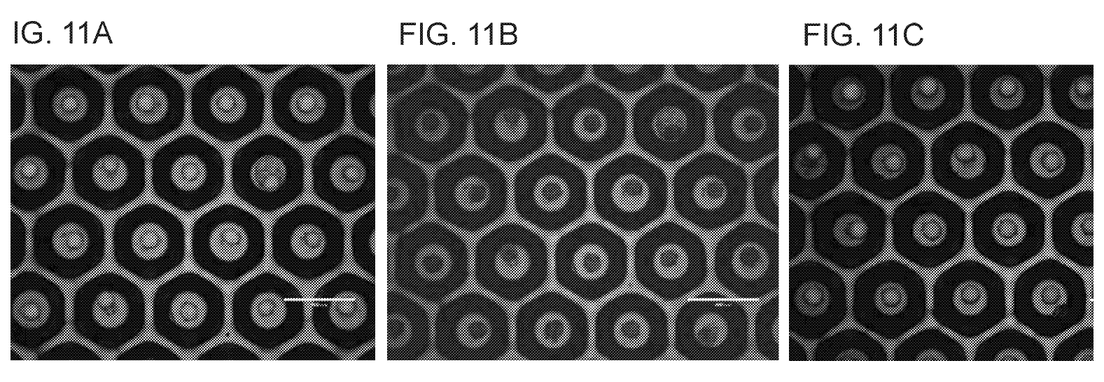

FIGS. 11A-C show images of HT-29 spheroid cultures (at 2x magnification) for the 0.5% AGR-L/0.7% Mc formulation. FIG. 11A shows images of spheroid cultures after 4° C storage during which the spheroids remained in place. FIG. 11B shows images after 99% of the cell storage and transport medium was removed from the culture vessel after three dilution cycles, with minimal spheroid loss. FIG. 11C shows an image of spheroid cultures 24 hr after removal of cell storage and transport medium that are in a recovery period.

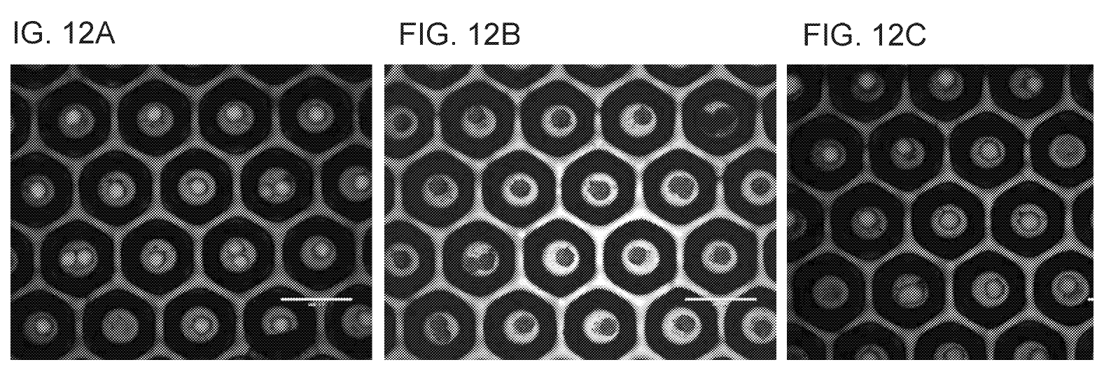

FIGS. 12A-C show images of HT-29 spheroid cultures (at 2x magnification) for the 0.5% AGR-L/0.5% Mc formulation. FIG. 12A shows images of spheroid cultures after 4° C storage indicating that some spheroids had been dislodged from the microcavities. FIG. 12B shows images after 99% of the cell storage and transport medium was removed from the spheroid culture vessel after three dilution cycles, with minimal spheroid loss, again showing dislocation of some spheroids FIG. 12C shows an image of cultures 24 hr after removal of cell storage and transport medium where the cell spheroids are in a recovery period. Trace amounts of the cell storage and transport media was observed in microcavities and many of the microcavities appear empty due to spheroid dislocation.

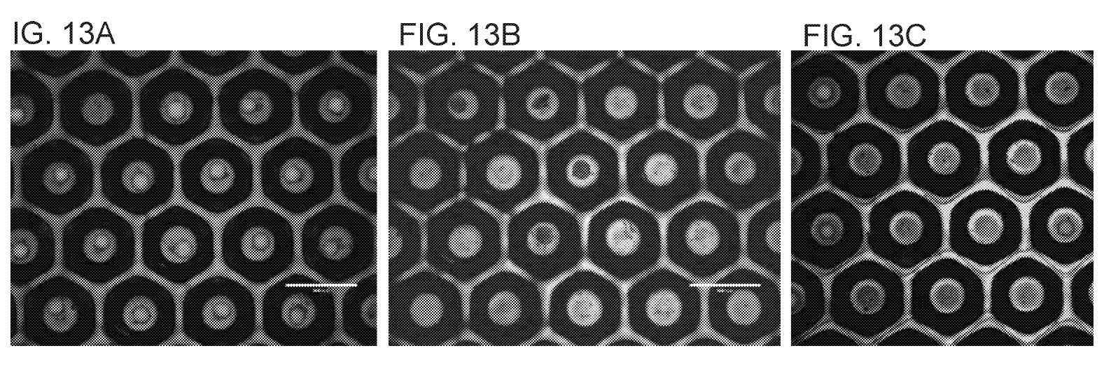

FIGS. 13A-C show images of HT-29 spheroid cultures (at 2x magnification) for the 0.5% AGR-L/0.35% Mc formulation. FIG. 13A shows images of cultures after 4° C indicating that some spheroids had been dislocated from the microcavities. FIG. 12B shows images after 99% of the cell storage and transport medium was removed from the spheroid culture vessel after three dilution cycles, with significant spheroid loss. FIG. 12C shows an image of a spheroid culture 24 hr after removal of cell storage and transport medium that are in a recovery period where many of the microcavities appear empty due to spheroid dislocation.

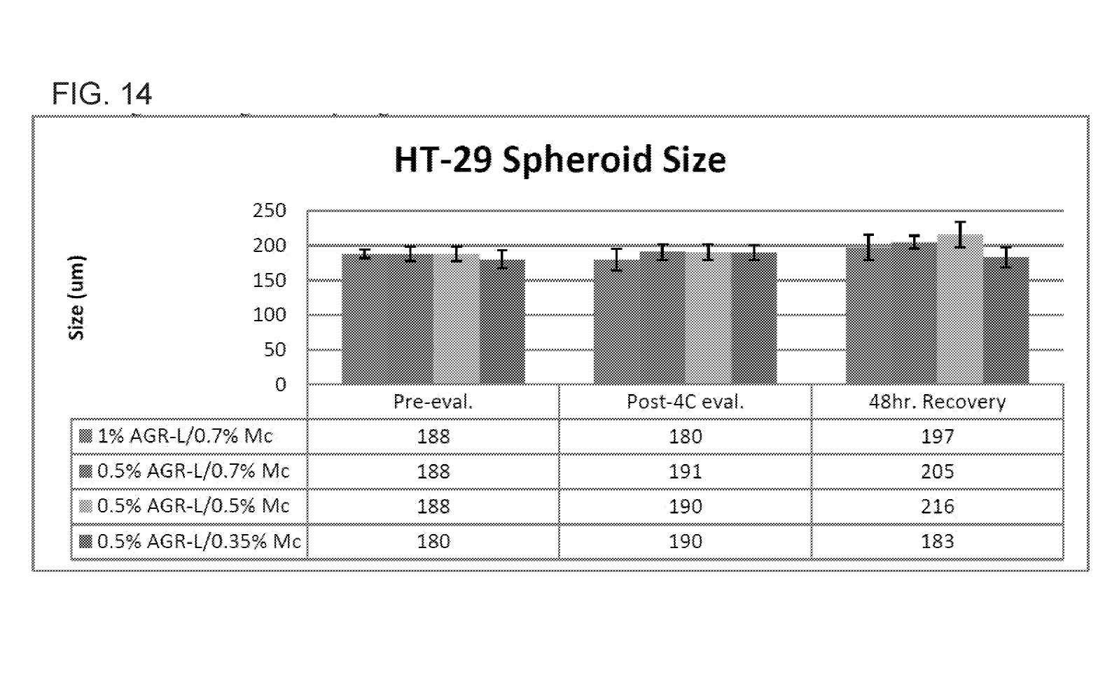

FIG. 14 shows the results related to spheroid health. Spheroid cell health was monitored through spheroid growth (size measurements). Size measurements were taken before cell storage and transport medium formulations addition, after storage at 4° C and 48 hr. post ship test evaluation including removal of cell storage and transport medium formulations. Measurements (FIG. 14) indicate no negative side effects (spheroid dissociation or change in size) after cold storage or during the recovery stage.

### Notes/Observations

0.35% Mc concentration is too low, it does not effectively prevent spheroid dislocation during transport evaluation.

0.5% AGR-L cell storage and transport medium formulations with 0.7% Mc and 0.5% Mc concentrations were similar to control in spheroid retention, but easier to remove from culture.

### Conclusions/Summary

Evaluations were done to determine acceptable concentration ranges for the agarose and methylcellulose components of the instantly-disclosed cell storage and transport medium.

Previous work established 1.0% to 0.5% as an acceptable working range for the agarose component. A current, and control, formulation is 1% AGR-L/0.7% Mc in IMDM

The 1% Mc concentration was too difficult to remove from culture.

The 0.35% Mc condition showed the highest spheroid loss during evaluation and was the least stable.

Methylcellulose (Mc) ranges between 0.7% and 0.5% work well at maintaining spheroids in place during the transportation test, were easy to work with, and easy to remove from the spheroid culture after evaluation.

Based on the above, cell storage and transport medium formulations ranges include 1.0 to 0.5% AGR-L, and 0.7 to 0.5% Mc.

### Example 2

### Storage Test

Viability assessment of cells in the spheroids that went through a storage test in the cell storage and transport medium was determined. The assessments were conducted with HT-29 cells (human colon cancer). The cell storage and transport medium included ultra-low gel temperature agarose (AGR-L, Sigma product # A5030), and methylcellulose (R&D Systems product #HSC006), with the gelling properties of both modified to provide the ideal conditions for transporting cells as spheroids. The AGR-L is made at a 2% concentration in water, then diluted to 1% with 2x concentrated cell culture medium to maintain proper media component concentrations. AGR-L becomes 0.5% when mixed 1:1 with the methylcellulose media. The methylcellulose is used as received at a 1.4% stock solution in medium (IMDM) which becomes diluted to 0.7% when mixed with the AGR-L. A 1:1 ratio of methylcellulose to agarose enables gel formation at 4 degrees C, and return to a viscous liquid state at 37 degrees C. The mixture also exhibits some properties of a non-Newtonian fluid, in that if the mixture seems to be solid at 37 degrees C, exerting a shear force on the mixture (by tapping the container), will cause the mixture to liquefy. This property is commonly referred to as “shear-thinning”.

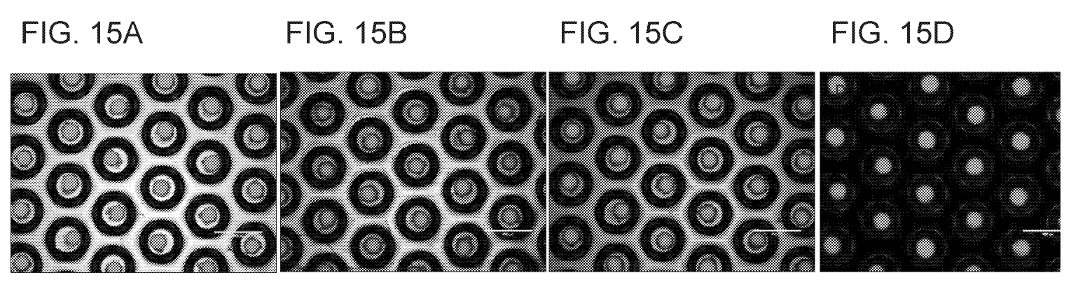

FIGS. 15A-D show the results of the storage test, which are images of HT-29 cells labeled with green fluorescent protein (GFP), that had formed spheroids. FIG. 15A shows the spheroids in normal cell culture growth medium of McCoy’s with 10% FBS. FIG. 15B shows the cells in cell storage and transport medium (0.5% ultra-low gel temperature agarose/0.7% R&D Systems IMDM methylcellulose medium) after storage at 4° C for 24 hours. FIG. 15C shows the cells 24 hours after the cell storage and transport medium has been removed and replaced with growth medium. FIG. 15D shows the cells 48 hours after the cell storage and transport medium has been removed and the cells have been stained with propidium iodide to detect cell death. Dead cells would appear red, but no red color is detected. This viability assessment data demonstrates that cells in the spheroids can be successfully stored using the instantly-disclosed cell storage medium, systems, and methods.

### Example 3

### Mock Transportation Test

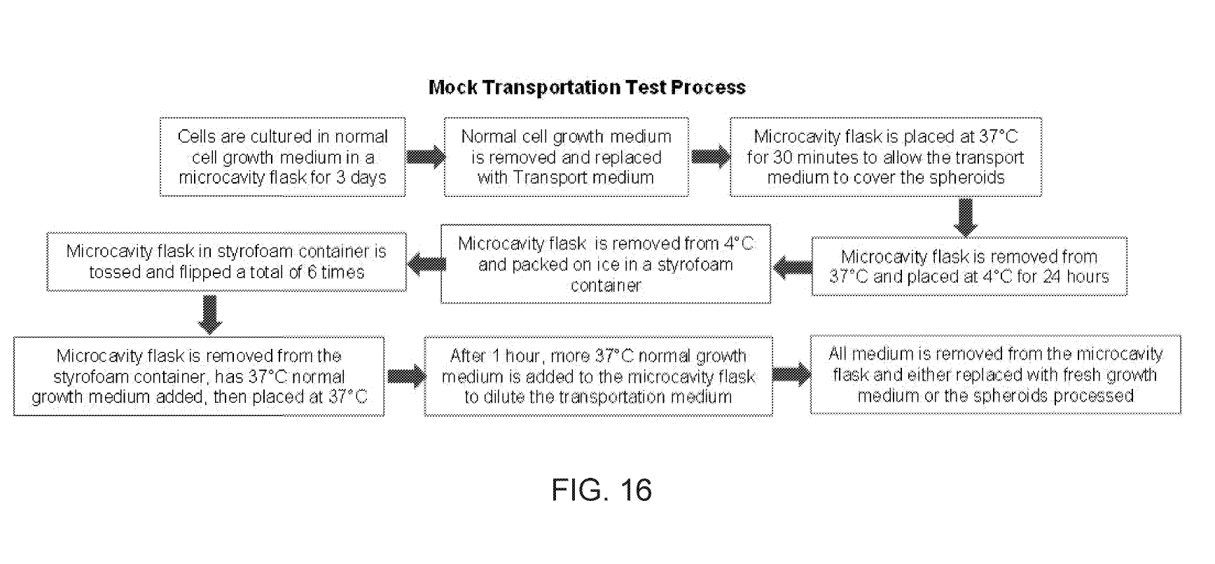

Viability assessment of cells in the spheroids that went through a mock transportation test in the cell storage and transport medium was determined. The transport media experiment was performed as follows and outlined in FIG. 16: 1) Normal culture medium is removed and replaced with transport medium. 2) The microcavity culture vessel is placed at 37° C for one hour. 3) The microcavity culture in transport medium is moved to 4° C for 24 hours. 4) The test vessels are packed into a Styrofoam box with ice packs to keep transport medium in solid state. 5) The Styrofoam box containing the microcavity vessel with the spheroids in transport medium is tossed and shaken to mimic transportation conditions. 6) The microcavity vessel is removed from the Styrofoam box and placed in the hood where 37° C culture medium is added.7) The microcavity vessel is placed at 37° C for one hour or until the medium in the microcavity vessel is thin enough to remove without disturbing the spheroids. 8) The diluted transport medium is removed and replaced with fresh culture medium. 9) The microcavity vessel is returned to the incubator for 24 hours before viability of the cells in the spheroids is assessed. 10) Spheroids were dissociated into single cells using trypsin/EDTA, and viability obtained using a NucleoCounter. For comparison, the control used cells from spheroids that did not undergo transport testing. Viability comparison between cells in a mock transportation test using cell storage and transport medium of the instant disclosure demonstrated good viability (data not shown), demonstrating that cells in the spheroids can be successfully stored and transported using the instantly-disclosed cell storage medium, systems, and methods.

*with or without serum, other listed and known mediums

All publications and patents mentioned in the above specification are herein incorporated by reference. It will be apparent to those skilled in the art that various modifications and variations can be made to the present inventive technology without departing from the spirit and scope of the disclosure. Although the disclosure has been described in connection with specific preferred embodiments, it should be understood that the disclosure as claimed should not be unduly limited to such specific embodiments. Since modifications, combinations, sub-combinations and variations of the disclosed embodiments incorporating the spirit and substance of the inventive technology may occur to persons skilled in the art, the inventive technology should be construed to include everything within the scope of the appended claims and their equivalents.

What is claimed is:

1. A method for transporting cells comprising transporting at least one cell storage and transportation system, the at least one cell storage and transportation system comprising:
cells;
a cell culture article including a chamber comprising an array of microcavities; and
a cell storage and transport medium disposed in one or more of the microcavities and comprising a mixture of cell culture medium, agarose and methylcellulose, wherein a final agarose concentration in the cell storage and transport medium is about 0.5 to about 1.0% and a final methylcellulose concentration in the cell storage and transport medium is about 0.5 to about 0.7%;
wherein the cells are suspended in the cell storage and transport medium;
wherein each microcavity is structured to constrain the cells to grow in a three-dimensional spheroid conformation.

2. The method of claim 1, wherein the cells remain in place within the cell storage and transport medium when the at least one cell storage and transportation system is subjected to a drop test.

3. The method of claim 2, wherein the cell storage and transport medium is in a solidified form.

4. The method of claim 1, wherein each microcavity comprises:
a top aperture; and
a liquid impermeable bottom comprising a bottom surface, wherein at least a portion of the bottom surface is non-adherent to cells or comprises an agent that reduces cell adhesion to the bottom surface.

5. The method of claim 4, wherein the liquid impermeable bottom comprising the bottom surface is gas-permeable.

6. The method of claim 1, wherein the cell culture article comprises from 1 to about 2,000 chambers, wherein each chamber is physically separated from any other chamber.

7. The method of claim 1, wherein the agarose is an ultra-low gelling temperature agarose.

8. The method of claim 7, wherein the ultra-low gelling temperature agarose has a gelling temperature from 8° C to 17° C.

9. The method of claim 1, wherein:
the cell storage and transport medium is a firm gel at 4° C;
the cell storage and transport medium is a soft gel at 23° C; and/or
the cell storage and transport medium is a viscous liquid at 37° C.

10. The method of claim 1, wherein the cells are transported for a transport time of not more than 48 or 72 hours.

11. The method of claim 1, further comprising recovering the transported cells.

12. The method of claim 11, wherein recovering the transported cells comprises removing the cell storage and transport medium and replacing it with a cell culture medium.

13. The method of claim 11, wherein recovering the transported cells comprises:
incubating the cell culture article at about 37° C for at least about 1 hr; and
subsequently removing the cell storage and transport medium and replacing it with a cell culture medium.

14. The method of claim 11, wherein recovering the transported cells comprises:
adding a first cell culture medium that is about 37° C to the cell storage and transport medium;
incubating the cell culture article at about 37° C for at least about 1 hr; and
removing the cell storage and transport medium and replacing it with a second cell culture medium.

15. The method of claim 11, wherein recovering the transported cells comprises:
incubating the cell culture article at about 37° C for at least about 1 hr;
subsequently removing the cell storage and transport medium; and
extracting the cells from the cell culture article.

16. A method for storing cells in preparation for transport of the cells, the method comprising:
culturing cells in a cell culture article, wherein the cell culture article comprises a chamber, the chamber comprising an array of microcavities, each microcavity structured to constrain the cells to grow in a three-dimensional spheroid confirmation;
adding a cell storage and transport medium to each of the microcavities, the cell storage and transport medium comprising a mixture of cell culture medium, agarose and methylcellulose, wherein a final agarose concentration in the cell storage and transport medium is about 0.5 to about 1.0% and a final methylcellulose concentration in the cell storage and transport medium is about 0.5 to about 0.7%; and
solidifying the cell storage and transport medium.

17. The method of claim 16, wherein the cell storage and transport medium is added to each of the microcavities at about 37° C.

18. The method of claim 16, wherein the cell storage and transport medium is solidified at a temperature of about 4° C or less.

19. The method of claim 16, further comprising sealing the cell culture chamber.

20. The method of claim 16, wherein the cells remain in place within the solidified cell storage and transport medium when the cell culture article is subjected to a drop test.
